(19) United States
(12) Patent Application Publication
(10) Pub. No.: US 2018/0264061 A1
(43) Pub. Date: Sep. 20, 2018

(54) Methods of Treating Diarrhea in Companion Animals

(71) Applicant: JAGUAR ANIMAL HEALTH, San Francisco, CA (US)

(72) Inventor: Serge MARTINOD, Groton, CT (US)

(21) Appl. No.: 15/541,513

(22) PCT Filed: Jan. 8, 2016
(86) PCT No.: PCT/US2016/012681

§ 371 Date: Jul. 5, 2017

Related U.S. Application Data
(60) Provisional application No. 62/117,927, filed on Feb. 18, 2015.
(60) Provisional application No. 62/101,663, filed on Jan. 9, 2015.

(51) Int. Cl. A61K 36/47; A61K 9/00; A61P 1/12

(52) U.S. Cl. CPC A61K 36/47; A61P 1/12; A61K 9/0056

(57) ABSTRACT

Provided are methods and compositions for treating diarrhea, particularly secretory diarrhea, watery diarrhea and chemotherapy induced diarrhea (CID), in young and adult non-human companion animals, such as dogs and cats, with a therapeutically effective amount of a proanthocyanidin polymer from Croton lechleri, in either enteric or non-enteric form.

### FIELD OF THE INVENTION

The invention relates to the treatment of various forms of diarrhea, such as watery diarrhea resulting from different conditions, pathologies, or therapies, in companion animals, such as dogs, cats and other animal pets. The treatment methods involve the use of enteric or non-enteric formulations and forms of a proanthocyandin polymeric composition isolated from the plant Croton spp. or Calophyllum spp., or with a latex, extract, or food supplement derived therefrom. More particularly, the composition is effective in treating secretory or watery diarrhea caused by, or secondary to, cancer treatment or therapy (chemotherapy-induced diarrhea or CID), thereby reducing the severity and duration of the diarrhea in companion animals, particularly dogs, as well as increasing their tolerance to and recovery from treatment.

### BACKGROUND OF THE INVENTION

Diarrhea is one of the most common reasons for veterinary office visits for companion animals, such as dogs and cats, and is the second most common reason for visits to the veterinary emergency room, yet there are no FDA-approved anti-diarrheal or secretory products for the treatment of diarrhea in canine and feline companion animals. Annually in the United States, it is estimated that veterinarians handle approximately 6 million cases of acute and chronic watery diarrhea in dogs, approximately two-thirds of which are acute watery diarrhea.

In addition, as of 2012, there were approximately 74 million cats in the United States. Estimates indicate that veterinarians see approximately 2.9 million annual cases of acute and chronic watery diarrhea in cats, approximately two-thirds of which are acute watery diarrhea. Veterinarians typically treat watery diarrhea in cats with the same treatments used for dogs, namely antibiotics, probiotics, dietary restrictions and products approved and formulated for humans, such as Imodium and other anti-motility agents, as well as binding agents that absorb water, such as Kaopectate and Pepto-Bismol. However, the present treatments options are quite limited, because none address the water loss associated with watery diarrhea.

Secretory diarrheas, also called watery diarrheas, are a major source of illness and mortality in both young and adult non-human animals and are characterized by the loss of both fluid and electrolytes through the intestinal tract, leading to serious and often life-threatening dehydration. Secretory diarrhea is caused by a variety of bacterial, viral and protozoal pathogens and also results from other non-infectious etiologies such as ulcerative colitis, inflammatory bowel disease, environmental and stress conditions, cancers and neoplasias of the gastrointestinal tract, as well as chemotherapeutic treatments used in affected animals. All types of diarrheal disease may have a secretory component.

There are no currently no FDA-approved anti-secretory agents that directly address the water loss associated with watery diarrhea in companion animals such as dogs. Current treatments for watery diarrhea include oral rehydration solution, or ORS, anti-motility agents, absorbents and antibiotics. However, each of these approaches has known limitations. While ORS replaces the water loss associated with diarrhea, it can often extend the duration and severity of diarrhea in the animal. Anti-motility agents, which typically work by the mechanism of constipation, or by temporarily paralyzing normal intestinal contractions or peristaltic activity, are contraindicated for chronic use and are therefore inappropriate for certain, chronic diarrheal conditions. Anti-motility agents can also cause pain, cramping, and rebound diarrhea. Absorbents simply attempt to absorb toxin in the gut, often causing additional pain and cramping, and do not directly address the water loss. Antibiotics attempt to treat an infectious agent producing the toxin, but do not directly address water loss and carry a risk of altering gut flora, which alteration itself can cause diarrhea. The prevalent use of antibiotics has also come under increased scrutiny by the FDA as a result of problems associated with antibiotic resistance.

It is estimated that there are over 230,000 dogs receiving chemotherapy treatment for cancer each year in the United States, with over 25% suffering from chemotherapy-induced diarrhea or CID. Severe diarrhea is a frequent side effect of the most commonly administered chemotherapy drugs. If left untreated, CID in dogs can result in effects that are similar to those experienced by humans, for example, fluid and electrolyte losses, which can cause dehydration, electrolyte imbalance and renal insufficiency; nutritional deficiencies from alteration of gastrointestinal transit and digestion; and increased risk of infectious complication. Efficacy of the underlying cancer treatment may also be jeopardized if CID severity requires reductions in the absorption, frequency and/or dosage of chemotherapy. From the dog owner's perspective, there are significant practical implications of CID in dogs that may affect living arrangements, as well as the cost, time and attention required to clean and care for the dog and its surroundings on a daily basis. Veterinarians sometimes prescribe drugs used by humans in an effort to treat CID in dogs; however, supporting clinical information with respect to efficacy or dosing are lacking. In addition, administering a potentially unpalatable human formulation to a dog is often difficult and may lead to additional uncertainty surrounding the amount actually ingested by the animal.

The available and commonly used treatments for the several types of diarrhea that affect companion animals typically involve vital fluid replacement and electrolyte replenishment to counter or stop fluid and electrolyte loss. Other types of treatments include the administration of oral antibiotics and non-steroidal anti-inflammatory drugs. Depending on the cause(s), timing, severity and course of diarrhea and/or its associated disease or condition, the various known treatments may or may not be effective, and the animals may or may not respond adequately or in a timely manner, leading to premature death in many cases.

Because the economic and humane impacts of diarrhea and its related conditions on the afflicted companion animals, their handlers and caregivers are so great, there is a compelling need for alternative, safe, and medically effective, as well as cost effective, treatments and remedies for diarrhea and its associated symptoms in companion animals, such as dogs and cats, in particular. The present invention advantageously addresses such needs.

### SUMMARY OF THE INVENTION

The present invention provides a method of treating various forms of diarrhea, such as watery diarrhea, general acute watery diarrhea, or chemotherapy-induced diarrhea (CID), in non-human companion animals. In embodiments, the companion animals are dogs and cats. In a particular embodiment, the method is directed to treating forms of diarrhea, e.g., watery diarrhea, general acute watery diarrhea, or CID, in dogs. The method involves administering to an animal in need thereof a pharmaceutically acceptable composition comprising an aqueous soluble proanthocyanidin from Croton lechleri, wherein the composition is formulated in an effective amount to treat diarrhea in the animal. In an aspect, the methods effectively treat the symptoms associated with the diarrhea in afflicted animals; the symptoms may include, without limitation, dehydration, weight loss and electrolyte loss. Some of these symptoms can manifest quickly, have long term health implications for a companion animal and even result in death. In an embodiment, the diarrhea is episodic diarrhea. In an embodiment, the diarrhea is acute diarrhea.

In an aspect, the present methods are useful for treating general acute watery diarrhea in companion animals in need thereof, as well as diarrhea resulting from chemotherapy treatment of animals undergoing such treatment. Although dogs are especially amenable to the methods described herein, the use of such methods for treating various forms of diarrhea, e.g., watery diarrhea, in other companion animals, or pets, such as cats, small rodents (pocket rodents), is not intended to be particularly limiting. In its various aspects, the methods are useful for treating and/or preventing various forms of diarrhea in companion animals, e.g., dogs and cats, and in animals at risk of becoming afflicted with a diarrhea condition.

In an aspect, the present methods are useful for treating chemotherapy-induced diarrhea (CID) in companion animals, particularly dogs, that are undergoing treatment or therapy for various types of cancers. Cancers in dogs are, on average, quite common, especially in older animals. For example, fifty percent of dogs over the age of ten often develop cancer at some point. Typical cancers in dogs include, without limitation, malignant lymphoma, e.g., in the lymph nodes; skin cancers and tumors, such as mast cell tumors; mammary gland tumors; soft tissue sarcomas; melanomas, and bone cancers, e.g., osteosarcomas and osteocarcinomas. With better care of companion animals and pets, the animals' lifespans increase and cancers may be more readily detected and treated. The present compositions and methods provide treatments for diarrhea that is associated with cancer therapy and treatments of companion animals, such as dogs, for example, chemotherapy or radiation treatments, depending upon the cancer type.

The present method and anti-diarrheal products generally act by normalizing the flow of ions and water in the intestinal lumen, the dysregulation of which is the last step common to the manifestation of watery diarrhea. As a result, such methods and products may be effective in addressing watery diarrhea, regardless of cause. In addition, the channels that regulate the flow of ions and water, including channels known as CFTR and CaCC (the sites of action of the gastrointestinal compositions and products described herein), are generally present in mammals. Thus, the compositions and products of the invention are expected to be effective in preventing the debilitating and devastating ramifications of watery diarrhea in companion animals, such as typical warm-blooded, non-human pets.

In embodiments of the method of the invention, the composition, formulation, or product is a pharmaceutically acceptable C. lechleri proanthocyanidin composition that is an enteric or enteric coated formulation. In other embodiment, the pharmaceutically acceptable C. lechleri proanthocyanidin composition is an non-enteric formulation. In embodiments of the method, the C. lechleri proanthocyanidin polymer composition is SB 300, SP 303, or crofelemer. In an embodiment, the non-human animal is a young or juvenile companion animal. In an embodiment, the non-human animal is an adult companion animal. The method of the invention is not particularly limited as to the species of the companion animal and can include, for example, canine, feline and rodent species as described herein. In particular embodiments, the companion animal is a dog, a puppy, a cat, or a kitten. It will be understood that, as used herein, the terms ‘composition’ and ‘formulation’ may be used interchangeably.

In other embodiments of the above method the C. lechleri proanthocyanidin polymer or a composition comprising the C. lechleri proanthocyanidin polymer is administered to the animal in an amount of at least 1 mg to 500 mg, or in an amount of 5 mg to 250 mg; or in an amount of 10 mg to 200 mg. In an embodiment, the C. lechleri proanthocyanidin polymer, or a composition comprising the C. lechleri proanthocyanidin polymer, is administered to the animal in a treatment dose of 20 mg to 100 mg. In an embodiment, the C. lechleri proanthocyanidin polymer, or a composition comprising the C. lechleri proanthocyanidin polymer, is administered to the animal in a treatment dose of 20 mg to 50 mg, e.g., 40 mg. In an embodiment, the C. lechleri proanthocyanidin polymer, or a composition comprising the C. lechleri proanthocyanidin polymer, is administered to the animal in a treatment dose of 40 mg. In an embodiment, the C. lechleri proanthocyanidin polymer, or a composition comprising the C. lechleri proanthocyanidin polymer, is administered to the animal in a treatment dose of 60 mg to 100 mg, e.g., 80 mg. In an embodiment, the C. lechleri proanthocyanidin polymer, or a composition comprising the C. lechleri proanthocyanidin polymer, is administered to the animal in a treatment dose of 80 mg. In still other embodiments of the method, the C. lechleri proanthocyanidin polymer composition is administered as powder reconstituted with a liquid selected from oral electrolytes, milk, milk replacer, physiological saline, or water. In other embodiments of the method, the C. lechleri proanthocyanidin polymer composition is administered as a bolus, such as a pill, tablet or capsule. In an embodiment, the C. lechleri proanthocyanidin polymer composition is orally administered. In an embodiment, the C. lechleri proanthocyanidin polymer composition is in a chewable solid form, e.g., pill, tablet, capsule, or other solid form, that is orally administered to a companion animal in need, especially dogs and cats. In an embodiment, the C. lechleri proanthocyanidin polymer composition is provided in a soft, chewable formulation, also called a soft chewable form herein, that is orally administered to a companion animal in need, especially dogs and cats. The chewable solid form or soft chewable form is typically flavored, e.g., a beef flavor, chicken flavor, or fish flavor, for example, for palatability to the animal to which it is administered. The chewable or soft chewable solid dosage form may be molded into a shape, size, and/or configuration or conformation that is suitable and appropriate for oral administration to the companion animal. In addition, the C. lechleri proanthocyanidin polymer composition may be administered in an animal's food or drink.

In embodiments of the method, the C. lechleri proanthocyanidin polymer composition may be in a variety of forms particularly suitable for the animal undergoing treatment, such a dog. For example, the composition may be in the form of a gel, a paste, or gel paste, which may be administered to the animal by topical application, for example, on the roof of the animal's mouth. In some cases, the C. lechleri proanthocyanidin polymer-containing composition includes pharmaceutically acceptable ingredients and/or is produced in a manner that provides a product having a consistency or texture that can be chewed, chomped, or gnawed by the animal. In a particular case, the C. lechleri proanthocyanidin polymer-containing composition includes pharmaceutically acceptable ingredients and/or is produced or formulated in a manner that provides a product having a chewable consistency or texture, e.g., a soft or semi-soft chewable formulation. In some cases, the composition may be contained in a delivery device, which can be a syringe, such as a needle-less syringe. In some embodiments of the method, composition in various forms comprises polymeric microparticles or nanoparticles containing the C. lechleri proanthocyanidin polymer composition. In an embodiment, the polymeric microparticles or nanoparticles are pH-sensitive.

In a particular embodiment, the C. lechleri proanthocyanidin polymer composition, also referred to as a C. lechleri proanthocyanidin polymer formulation or product herein, is an orally administered, chewable, flavored formulation for the treatment of diarrhea, such as CID, in dogs. In a particular embodiment, the chewable formulation is a solid form such as, without limitation, a tablet, pill, or capsule. In a particular embodiment, the chewable formulation is solid form that is soft or semi-soft and can be readily masticated and swallowed by the animal. In a particular embodiment, the chewable solid form contains flavoring or flavored substances so as to be more palatable to the animal to which it is orally administered. In an embodiment, the chewable solid form is beef-flavored or flavored to the taste of a particular animal to be treated, such as a fowl, fish, or vegetable flavor. In another embodiment, the chewable solid form comprising a C. lechleri proanthocyanidin polymer formulation, e.g., SP 303 or SB 300, is orally administered once or twice per day a dosage amount of 0.1 mg/kg to 50 mg/kg; or in an amount of 0.5 mg/kg to 30 mg/kg; or in an amount of 0.5 mg/kg to 25 mg/kg. Other more specific dose ranges may be from 0.1 mg/kg to 0.5 mg/kg; or from 0.1 mg/kg to 0.3 mg/kg; or from 0.2 mg/kg to 0.3 mg/kg; or from 0.4 mg/kg to 1.5 mg/kg; or from 0.8 mg/kg to 10 mg/kg; or from 1 mg/kg to 4 or 5 mg/kg. Other dosage amounts may be about 0.1 mg/kg; 0.2 mg/kg; 0.3 mg/kg; 0.4 mg/kg 0.5 mg/kg; 0.6 mg/kg; 0.7 mg/kg; 0.8 mg/kg; 1 mg/kg; 2 mg/kg; 3 mg/kg; 4 mg/kg; or 5 mg/kg, and the like, depending on the weight or body mass of the companion animal. In a particular embodiment, the chewable solid form is a beef-flavored soft chewable form of the C. lechleri proanthocyanidin polymer composition, which is orally administered in a dosage amount of 2 mg/kg or 4 mg/kg for an animal, such as a canine animal, twice per day. In another embodiment, the C. lechleri proanthocyanidin polymer composition is administered for three days. In another embodiment, the C. lechleri proanthocyanidin polymer composition is SP-303.

By way of example, dogs can generally weigh between 2 kg and 80 kg. For dogs, the dose of C. lechleri proanthocyanidin polymer or polymer composition, e.g., SP 303 or SB 300, may range from 0.5 mg/kg to 25 mg/kg, more particularly, 2 mg/kg, and may be adjusted as appropriate according to the body weight of the animal. For other companion animals of different weights and body mass, the doses may be adjusted accordingly.

In an embodiment, the C. lechleri proanthocyanidin polymer composition is enteric coated for targeted release of the active pharmaceutical ingredient (API) in the intestine. In an embodiment, the active ingredient or API is crofelemer, e.g., SP 303. In an embodiment, the active ingredient is present in a dosage amount of 1 mg to 100 mg; or 10 mg to 50 mg, or in a dosage amount of 20 mg to 50 mg, or alternatively, particularly for larger dogs (of greater than 20 kg), a dosage amount of 60 mg to 100 mg. In a particular embodiment, the dosage amount is 40 mg of active ingredient and, in alternative embodiments, particularly for larger dogs, the dosage amount is 80 mg of active ingredient. In an embodiment, the dose is a reconstituted suspension containing enteric coated C. lechleri proanthocyanidin polymer microparticles (e.g., 40 mg of active SP 303) in an amount of 4 mg/mL in 10 mL for an approximately 20 kg companion animal such as a dog. See, e.g., Examples 1, 2, 3 and 5. In an embodiment, the enteric coated product comprises microparticles or nanoparticles containing the C. lechleri proanthocyanidin polymer composition or botanical extract of C. lechleri. In an embodiment, the enteric coated product comprises microparticles or nanoparticles containing as active the C. lechleri proanthocyanidin polymer composition, which is SP 303, for dosing, e.g., in an amount of 2 mg/ml per day (or per dose, 2 times per day) for 3 days.

In an embodiment, the methods improve gut health and control diarrhea in the affected companion animals by providing a proanthocyanidin polymer from Croton lechleri in an effective amount to control or treat the diarrhea in the treated companion animals. In an embodiment the proanthocyanidin polymer is a formulation, composition, or extract from Croton lechleri. In an embodiment, the proanthocyanidin polymer from Croton lechleri is a more highly purified composition containing proanthocyanidin polymer or oligomer, such as crofelemer or SB 300 compositions described herein. In an embodiment the proanthocyanidin polymer is a formulation, composition, or botanical extract from Croton lechleri. In an embodiment, the formulation, composition, or botanical extract from Croton lechleri is in the form of a paste or gel. In a particular embodiment, the composition or formulation comprises beads (nano or microparticles) comprising enterically coated SB 300 or SP 303 and is orally administered to animals in need. In a particular embodiment, the composition or formulation is a chewable product and comprises beads (nano or microparticles) comprising enterically coated SB 300 or SP 303. In a particular embodiment, the enterically coated product is orally administered to a dog, or companion animal, once a day or twice a day. In a particular embodiment, the enterically coated product is orally administered to a dog, or companion animal, twice daily, for one day or for multiple days, e.g., 2, 3, 4, 5, 6, 7, 8, 9, or 10 days, or for two weeks. In some embodiments, the product is orally administered for multiple consecutive days, such as at least two or three consecutive days. In a specific embodiment, enteric coated microgranules of the C. lechleri proanthocyanidin polymer composition, such as crofelemer (e.g., SP 303), are orally administered to a companion animal such as a dog afflicted with diarrhea at a dose of 2 mg/kg per day or twice per day, or 4 mg/kg per day or twice per day. In an embodiment, the dose is administered for three days.

In an embodiment, the C. lechleri proanthocyanidin polymer composition, formulation, or product is provided in a method for treating chemotherapy-induced diarrhea (CID) in companion animals, especially dogs, as described herein. In a particular embodiment, the C. lechleri proanthocyanidin polymer composition is enteric coated, comprises nano or microparticles and is in a chewable solid oral dosage form.

### BRIEF DESCRIPTION OF THE FIGURES

FIG. 1 shows a bar graph of daily fecal score as determined in the study described in Example 3. Days are denoted on the x-axis; daily fecal score is indicated on the y-axis. In each of the groupings of the bars in the graph by day, the first of the three bars (left-most bar) indicates “enteric coated” treatment drug; the second of the three bars (middle bar) indicates “non-enteric coated” treatment drug; and the third of the three bars (right-most bar) indicates placebo treatment.

FIG. 2 shows a graph of percent responders versus treatment day according to the study described in Example 5. Treatment A indicates the treatment group that received the crofelemer SP-303 product, and Treatment B indicates the treatment group that received placebo during the study. As shown in FIG. 2, the response in the SP-303 treatment arm was greater than that in the placebo treatment arm on all days by at least 10%. In accordance with the study, a responder was a subject dog that had formed stools with no follow up unformed stool, day by day.

### DETAILED DESCRIPTION OF THE INVENTION

Diarrhea is an increase in the frequency, fluidity or volume of feces. It is a common problem in companion animals, such as dogs and cats, and it is characterized by duration (acute versus chronic), anatomic location and pathophysiologic mechanism. One of the most common pathophysiological mechanisms is secretory diarrhea caused by abnormal ion transport in intestinal epithelial cells. The most common causes of secretory diarrhea in dogs and cats are abnormal mediators that result in changes in intracellular cyclic adenosine monophosphate (cAMP), cyclic guanosine monophosphate (cGMP), calcium and/or protein kinases, which, in turn, cause an increase in chloride ion secretion. Such mediators include, without limitation, endogenous enteric hormones or neuropeptides, inflammatory cell products, bacterial endotoxins, laxatives, fatty acids, bile acids and various types of drugs.

Chemotherapy-induced diarrhea (CID) may occur in companion animals, particularly dogs, that are undergoing treatment or therapy for various types of cancers. The drugs, agents and compounds used to treat cancers may result in diarrhea as a side effect, similar to the situation in humans who are treated for cancer or tumors. Illustratively, diarrhea may result from chemotherapy or radiation treatment of companion animals, such as dogs, for cancers, such as lymphomas; malignant lymphoma; skin cancer and tumors, e.g., mast cell tumors; mammary gland tumors; soft tissue sarcomas; melanomas; and bone cancers, e.g., osteosarcomas and osteocarcinomas. Certain breeds of companion animals, such as dogs, especially those that are genetically inbred or those of certain species, have higher than normal incidences of specific cancers. Thus, cancers requiring the treatment methods and compositions of the invention may be genetically-based, but also may be due to one or more environmental or spontaneous causes. The present compositions and methods provide treatments for diarrhea that is associated with cancer therapies and treatments for companion animals, such as dogs receiving chemotherapy or radiation treatments.

The anti-diarrhea treatment methods and compositions described herein are not limited to CID related to a particular cancer type or cause, and may be advantageous in treating or preventing diarrhea stemming from various types, forms and combinations of chemotherapy and cancer treatments. A variety of chemotherapeutic drugs are available for the treatment of various types of cancers in companion animals such as dogs. The drugs may be used alone or in combination and may result in diarrhea, i.e., the passage of watery stools within a 24-hour period. Many of the same chemotherapeutic agents are used to treat cancers in human patients and may also cause diarrhea. Examples of chemotherapeutic drugs which may be associated with causing diarrhea in canine species undergoing treatment for cancer include, without limitation, actinomycin D, L-asparaginase, carboplatin, chlorambucil, cisplatin, cyclophosphamide, cytarabine, doxorubicin, lomustine, methotrexate, mitoxantrone, vinblastine, vincristine. L-asparaginase, carboplatin, cyclophosphamide, doxorubicin and vincristine are included among the most commonly used anticancer drugs.

Multi-drug protocols are frequently used as chemotherapy treatments in dogs. Some drug combinations that have been used for cancer treatment in dogs include, illustratively, vincristine/L-asparaginase/cyclophosphamide/doxorubicin/methotrexate/prednisolone; vincristine/doxorubicin; doxorubicin/cyclophosphamide/vincristine/prednisolone; vincristine/L-asparaginase/cyclophosphamide/doxorubicin/prednisolone; vincristine/cyclophosphamide/doxorubicin; doxorubicin/cyclophosphamide; cyclophosphamide/vincristine/prednisolone/L-asparaginase/doxorubicin; doxorubicin/carboplatin (osteosarcoma); prednisolone/L-asparaginase/vincristine/cyclophosphamide/doxorubicin/radiation (chemoradiotherapy).

Two chemotherapeutic drugs that are approved for use specifically in dogs include toceranib phosphate (PALLADIA), a tyrosine kinase inhibitor approved for the treatment of Grade 2 or 3 mast cell tumors; and mastinib (KINAVET-CA1® or MASTIVET), a tyrosine kinase receptor inhibitor approved for the treatment of non-resectable canine mast cell tumors.

Without wishing to be limited by theory, in canine animals, the direct action of cytotoxic drugs used in chemotherapy treatment on dividing cells in the gastrointestinal (GI) tract causes adverse effects usually within 1 to 5 days, and occasionally up to 10 days, after initiating treatment. A typical pattern involves anorexia on day 1, followed by vomiting and/or diarrhea on day 2-3. Symptomatic treatment is required to mitigate the dehydration effects associated with diarrhea. Such treatment commonly involves the administration of intravenous fluids, electrolytes, anti-emetics, gut protectants, acidity regulators and/or antibiotics. If GI toxicity results in epithelial damage, there is a greater risk of sepsis and neutropenia, as the gut mucosa becomes a less effective barrier to bacterial translocation.

There are currently no products on the market with anti-secretory properties that effectively treat diarrhea, regardless of the cause, in companion animals, such as dogs and cats. At present, the principal goals of symptomatic therapy include restoration and maintenance of fluid and electrolyte balance, dietary modifications and administration of antimicrobials and anti-helminthics, if warranted, to an afflicted animal. In view of the adverse effects attendant with chemotherapeutic drug administration, the present formulations and methods provide a needed, advantageous and effective class of drugs that reduces electrolyte and fluid losses associated with diarrhea and severe diarrhea.

As used herein, companion animals treated by the methods of the present invention are domesticated non-human animals, also known as pets, which are typically and primarily kept for a person's company, social or emotional enjoyment and/or therapy, or protection and which are not typically kept for performance, production, research, or agricultural value. Unless otherwise noted, the use of the term “animal” herein denotes non-human, warm-blooded mammals of a number of different species that are considered as companions or pets for humans. Illustratively, and without limitation, the methods of the invention are useful for treating and/or preventing various forms of diarrhea in companion animals such as dogs; cats; rabbits (housed rabbits); ferrets; rodents/pocket rodents such as mice, gerbils, hamsters, chinchillas, non-laboratory rats and guinea pigs. As a general guide and without limitation, body weights of some types of companion animals may fall into the following approximate ranges: dogs (2 kg-100 kg); cats (2 kg-20 kg); rabbits (0.4 kg-5 kg); ferrets (0.5 kg-2 kg); gerbils (40 g-130 g); hamsters (50 g-150 g); rats (200 g-700 g); and guinea pigs (100 g-1.2 kg). In a preferred embodiment, the companion animal treated for diarrhea using the methods and products of the invention is a dog. In another preferred embodiment, the companion animal treated for diarrhea using the methods and products of the invention is a cat.

In an embodiment, the formulation and product provided by the present invention for use in treating various types of watery diarrhea in dogs effectively acts at the last physiological step in the manifestation of watery diarrhea, regardless of cause, by normalizing ion and water flow in the intestinal lumen of the animal. In accordance with the invention, an optimal treatment for watery diarrhea would directly address water loss without causing constipation, affecting normal peristaltic activity, or altering normal body absorption of other drugs or normal physiological function of the gut. Addressing water loss associated with watery diarrhea through the methods of the invention offers an improvement in the quality of life of dogs and provides attendant benefits to the dog owner. The C. lechleri proanthocyanidin polymer compositions containing crofelemer or a botanical extract from C. lechleri, as well as the SB 300 and SP 303 compositions described herein, provide products that act by normalizing the flow of ions and water in the intestinal lumen, the dysregulation of which is the last step common to the manifestation of watery diarrhea. In addition, the channels that regulate this ion and water flow, including CFTR and CaCC channels, which are the sites of action of the gastrointestinal products of the invention, are generally present in mammals. As a result, such products may be effective in addressing watery diarrhea, regardless of cause, and stand to be effective in preventing the debilitating and devastating ramifications of watery diarrhea in companion animals.

In view of the unpleasant and debilitating effects of diarrhea in non-human animals, the methods of the present invention provide a new means to treat diarrhea in afflicted companion animals. The methods of the invention provide treatment of diarrhea resulting from a number of different sources and causes, for example, in dogs and cats, with an effective amount of a polymeric proanthocyanidin composition from a Croton species or Calophyllum species, or with a latex, extract or food supplement botanical extract derived therefrom. The effective treatment of diarrhea with a proanthocyanidin polymer composition from Croton lechleri, or with a latex, extract or food supplement botanical extract derived therefrom is advantageous in view of a dearth of such treatments for domestic, companion animals that suffer from diarrhea and related conditions.

Crofelemer, e.g., SP 303 described below, is a purified extract of a Croton species such as Croton lechleri containing one or more ingredients with potent anti-secretory properties and a unique mode of action. In accordance with the present invention, crofelemer and forms thereof are used in treating diarrhea in companion animals, such as dogs and cats, and other companion animal species. More specifically, the mechanism of action of polymeric proanthocyanidin compositions, or purified extracts such as crofelemer, is through the inhibition of both the cystic fibrosis transmembrane conductance regulator protein (CFTR) chloride ion channel and the calcium-activated chloride ion channels (CaCC). The polymeric proanthocyanidin composition acts by blocking chloride ion channel secretion and the accompanying high volume water loss occurring in diarrhea, thus normalizing the flow of chloride ions and water in the gastrointestinal (GI) tract.

The invention provides methods directed to treating the debilitating problem of watery diarrhea from various causes in young and adult non-human companion animals, such as dogs. The methods are effective in reducing and/or alleviating watery diarrhea or CID in such non-human animals in need thereof. In particular, the methods are directed to the treatment of diarrhea, particularly secretory/watery diarrhea, caused by any number of factors leading to diarrhea in companion animals. By way of example, an animal may suffer from diarrhea as a result of infection, such as bacterial infection, viral infection, parasitic/protozoan infection, (Giardia), environmental conditions, pancreatitis, chemotherapy-induced treatments, dietary indiscretions, intake of soil and unhealthy contaminants, and the like, alone, or in combination with other infectious agents, disease or environmental conditions, in adult and young companion animals. The invention further provides formulations and compositions highly suitable for treating diarrhea in young and adult dogs. Without wishing to be limiting, “young” or “juvenile” animals are generally a year old, or under one year of age, while “neonatal” animals are generally two weeks of age or less.

The present invention particularly relates to treating watery diarrhea, acute watery diarrhea, or CID in adult or young companion animals, such as dogs, with physiologically and pharmaceutically acceptable formulations and compositions comprising a therapeutically effective amount of an antidiarrheal agent comprising a proanthocyanidin polymer obtained from a Croton spp., preferably Croton lechleri (C. lechleri). The proanthocyanidin polymer composition can also be obtained from a Calophyllum spp., in particular Calophyllum inophylum. In an specific embodiment, the pharmaceutically acceptable composition comprises a proanthocyanidin polymer from Croton lechleri. In a particular embodiment, the proanthocyanidin polymer is enterically protected beads, including enteric beads including SB 300 or SP 303.

### Treatment of Chemotherapy-Induced Diarrhea (CID) in Dogs

In an particular embodiment, the present methods and products provide a needed treatment of chemotherapy-induced diarrhea (CID) in dogs. Severe diarrhea is a frequent side effect of the most commonly administered chemotherapy drugs. If left untreated, CID in dogs can result in effects that are similar to those experienced by humans, for example, fluid and electrolyte losses, which can cause dehydration, electrolyte imbalance and renal insufficiency; nutritional deficiencies from alteration of gastrointestinal transit and digestion; and increased risk of infectious complication. Efficacy of the underlying cancer treatment may also be jeopardized if CID severity requires reductions in the absorption, frequency and/or dosage of chemotherapy. Veterinarians sometimes prescribe drugs used by humans in an effort to treat CID in dogs; however, supporting clinical information with respect to efficacy or dosing are lacking. In addition, administering a potentially unpalatable human formulation to a dog is often difficult and may lead to additional uncertainty surrounding the amount actually ingested by the animal. Examples of types of chemotherapeutic drugs and drug combinations, as well as their effects following administration, have been set forth herein above.

In some embodiments, the methods, compositions, and formulations described herein may be prophylactically provided to an animal undergoing chemotherapeutic, including radiation, treatment for cancer to reduce, abrogate, abate, or eliminate the concomitant adverse effects and symptoms of diarrhea before they present and/or become too severe. Because diarrhea may ensue within 1-5 days, or within 2-3 days, following chemotherapy treatment, the treatment methods, compositions and formulations of the invention may be administered to an animal in need at a predetermined time prior to the animal's receiving chemotherapy treatment keeping in mind general times of diarrhea onset, based on the foregoing, as well as at the discretion of veterinary personnel. In addition, the treatment methods, compositions and formulations as described herein may be provided concomitantly with, or on the day that the animal receives chemotherapy treatment. In addition, the treatment methods, compositions and formulations may also be administered following the onset of diarrhea, for example, as soon as possible after symptoms appear, to counter the adverse effects, reduce electrolyte and fluid losses by the animal and/or restore proper electrolyte balance, hydration and fluid balance in the animal's system. It will be understood that an animal in need may receive the compositions and formulations of the invention one time or more than one time, e.g., two, three, four, or more times per day, whether administration occurs prior to, concomitantly with/on the same day as, or following the administration of one or more chemotherapy drugs.

For treating CID in dogs, a C. lechleri proanthocyanidin polymer composition, formulation or product, particularly designed for dogs, is provided by the present invention. Such a canine-palatable product constitutes an enteric coated C. lechleri proanthocyanidin polymer composition that is a flavored, e.g., beef-flavored, chewable, preferably a soft chewable, solid form, which is orally administered to a dog in need for the treatment of CID. In an embodiment, the product is administered to the animal in need twice daily. As described herein, the consistency and/or flavoring of the solid oral dosage form is not intended to be limiting. The C. lechleri proanthocyanidin polymer treatment product has a demonstrated anti-secretory mechanism of action; it acts locally in the gut and is minimally absorbed systemically. In addition, it does not alter gastrointestinal motility, has no significant effects on normally functioning intestinal ion channels and electrolyte or fluid transport; and has no side effects different from placebo. These features are further augmented by its lack of effect on the absorption and/or metabolism of co-administered chemotherapy drugs, orally or by other routes of administration. Without intending to be bound by theory, the product of the invention acts by normalizing the flow of excess ions and water in the intestinal lumen. The flow of excess ions and water into the intestinal lumen is the last step common to the manifestation of watery diarrhea. As a result, the product may be effective in the treatment of watery diarrhea associated with CID, or regardless of cause, for that matter. By way of example, following oral dosing for two or three days, the C. lechleri proanthocyanidin polymer composition (e.g., crofelemer or SP 303) together with ORS, can produce significant reduction in watery diarrhea, as demonstrated by the reduction of watery stool passage as well as reduced duration of diarrhea, urgency and dehydration.

In a particular embodiment related to CID treatment in dogs, the C. lechleri proanthocyanidin polymer composition is a chewable solid product, preferably, a soft chewable solid product that is beef-flavored for the treatment of diarrhea, such as CID, in dogs. In an embodiment, the soft chewable product is orally administered in an amount of 2 mg/kg, more particularly, twice per day. The C. lechleri proanthocyanidin polymer containing product is formulated as enteric coated microparticles as the active ingredient for targeted release in the dog's intestine. In an embodiment, the C. lechleri proanthocyanidin polymer active ingredient is crofelemer or SP 303. In a particular embodiment, the active ingredient is present in a dosage amount of 40 mg. In an embodiment, the enteric coated product comprises microparticles or nanoparticles containing the C. lechleri proanthocyanidin polymer composition or botanical extract of C. lechleri.

In general terms, “treating” an animal according to the present methods refers to achieving or obtaining a desired physiologic and/or pharmacologic effect, whether prophylactic, therapeutic, or both. As used herein “treating” or “treatment” can refer to ameliorating, preventing, inhibiting, reversing, attenuating, alleviating, abrogating, minimizing, suppressing, reducing, decreasing, diminishing, stabilizing, eradicating, curing, or eliminating the deleterious effects of a disease or condition, or the progression or worsening of the disease or condition. For example, successful treatment may involve alleviating one or more symptoms of a disease or condition, although not necessarily all of the symptoms, of the disease or condition, or attenuating the symptoms or progression of the disease or condition. Curing or eliminating the disease or condition from the animal is an optimal outcome of the practice of the methods of the invention. Treatment of companion animals according to the present methods may be therapeutic or prophylactic.

According to the invention, treatment of an animal in need thereof typically involves the use or administration of an effective amount or a therapeutically effective amount of a proanthocyanidin polymer or a proanthocyanidin polymer composition preferably from a Croton spp. particularly C. lechleri, either enteric or non-enteric. Effective amount refers to the quantity (amount) of the composition, and the like, that induces a desired response in the animal subject upon administration or delivery to the animal. Optimally, an effective amount produces a therapeutic effect in the absence of, or with little or virtually no, adverse effects or cytotoxicity in the animal. Alternatively, any adverse effects associated with an effective amount are optimally outweighed by the therapeutic benefit achieved.

The treatment methods of the invention are directed to ameliorating, preventing, inhibiting, reversing, attenuating, alleviating, abrogating, minimizing, suppressing, reducing, decreasing, diminishing, stabilizing, eradicating, curing, or eliminating diarrhea and/or its associated symptoms that adversely affect the health, growth and survivability of young and adult companion animals. In an embodiment, the diarrhea is secretory/watery diarrhea. Such diarrhea can be a clinical sign of gastrointestinal (GI) disease in an animal; it can also reflect primary disorders outside of the digestive system, such as disorders affecting the large bowel or the small bowel. The methods described herein are also suitable for treating diarrhea resulting from infection, such as bacterial infection, in companion animals that may have comorbid conditions or disorders that cause diarrhea via different mechanisms involved in their pathogenesis, for example, osmotic diarrhea, secretory diarrhea, episodic diarrhea, or inflammatory and infectious diarrhea. In an embodiment, the young or adult companion animal may suffer from diarrhea associated with inflammation of the lining of the colon, such as colitis, or acute colitis, which can be caused by infection or inflammation of the bowel.

### Treatment of Other Forms of Diarrhea

Osmotic diarrhea is associated with absorption of water in the intestines, which depends upon adequate absorption of solutes. If excessive amounts of solutes are retained in the intestinal lumen, water will not be absorbed and diarrhea results. Osmotic diarrhea typically results from ingestion of a poorly absorbed substrate, for example, a carbohydrate or divalent ion or from malabsorption of any type, such as an inability to absorb certain carbohydrates. Secretory diarrhea occurs when the secretion of water into the lumen of the intestine exceed absorption. Under normal conditions, large volumes of water are secreted into the small intestinal lumen, but a large portion of this water is efficiently absorbed before reaching the large intestine.

Secretory diarrhea can result from exposure of an animal to toxins (enterotoxins) from certain types of bacteria, such as cholera toxin of Vibrio cholerae and heat-labile toxin of E. coli. Massive diarrhea is induced from such microorganisms as a consequence of their toxins strongly activating adenylyl cyclase, which causes a prolonged increase in the intracellular concentration of cyclic AMP within crypt enterocytes. This increase, in turn, results in prolonged opening of the chloride channels that contributes to secretion of water from the crypts, thereby allowing uncontrolled secretion of water. These bacterial toxins can also affect the enteric nervous system, resulting in an independent stimulus of water secretion.

Inflammatory and infectious diarrhea can be caused by the disruption of the epithelium of the intestine due to microbial or viral pathogens. Typically, the epithelium of the digestive tube is protected from insult by a number of mechanisms that constitute the gastrointestinal barrier. However, the gastrointestinal barrier can be breached and result in diarrhea. Destruction of the epithelium results not only in leaking of serum and blood into the lumen but also is often associated with significant destruction of adsorptive epithelium. When this occurs, the absorption of water becomes highly inefficient and diarrhea results. The pathogenic culprits frequently associated with infectious diarrhea include bacteria, such as E. coli, Campylobacter and Salmonella; viruses, such as rotaviruses, coronaviruses, parvoviruses and norovirus; and protozoa, such as coccidia species, Cryptosporium and Giardia. In addition, the response of the immune system to inflammatory conditions in the bowel contributes greatly to the development of diarrhea. Activated white blood cells are stimulated to produce and secrete inflammatory mediators and cytokines that stimulate secretion. An secretory component is thus imposed upon and exacerbates an inflammatory diarrhea. Moreover, reactive oxygen species produced by leukocytes can damage or destroy intestinal epithelial cells, which are replaced with immature cells that are generally lacking in the brush border enzymes and transporters necessary for the absorption of nutrients and water. Thus, components of an osmotic (malabsorption) diarrhea provide additional pathology and problems for an afflicted companion animal.

In some cases, diarrhea in companion animals results from a combination of the invasion of infectious bacteria and noninfectious factors. Frequently, noninfectious causes of diarrhea in young or adult companion animals are considered to be factors that may predispose or contribute to an animal's susceptibility to infectious agents and causes of diarrhea. Whether the cause of diarrhea in animals is infectious or noninfectious, the absorption of fluids from the intestine is altered and life-threatening electrolyte imbalances can occur. The affected animals lose fluids, rapidly dehydrate and suffer from electrolyte loss and acidosis. Although infectious agents may cause an initial damage to the animal's intestine, actual death from watery diarrhea (serious diarrhea) in animals usually is a consequence of dehydration, acidosis and loss of electrolytes, which may be difficult to replenish in adequate amount and time. Accordingly, the methods and formulations of the invention are suitable for treating various forms of diarrhea, e.g., secretory/watery diarrhea and CID, and the symptoms of diarrhea, such as dehydration and electrolyte loss, in an effort to reduce and/or eliminate the diarrhea and prevent more severe dehydration or even death of the companion animal.

Proanthocyanidins and Tannins Obtained from Plant Extracts

Proanthocyanidins are types of condensed tannins, which are found in a large number of plants and are classified as hydrolyzable or condensed. Tannins and, in particular, proanthocyanidins are contained in many plants used in traditional medicine as treatment or prophylaxis for diarrhea (See, e.g., Yoshida et al., 1993, Phytochemistry, 32:1033; Yoshida et al., 1992, Chem. Pharm. Bull., 40:1997; Tamaka et al., 1992, Chem. Pharm. Bull., 40:2092).

Proanthocyanidins are comprised of at least two or more monomer units that may be of the same or different monomeric structure. The monomer units (generally termed “leucoanthocyanidins”) are generally monomeric flavonoids which include catechins, epicatechins, gallocatechins, galloepicatechins, flavanols, flavonols, flavan-3,4-diols, leucocyanidins and anthocyanidins. The polymer chains are thus based on different structural units, creating a wide variation of polymeric proanthocyanidins and a large number of possible isomers (Hemingway et al., 1982, J. C. S. Perkin, 1:1217). Larger polymers of the flavonoid 3-ol units are predominant in most plants and often have average molecular weights above 2,000 daltons (Da), containing 6 or more units (Newman et al., 1987, Mag. Res. Chem., 25:118).

Proanthocyanidin polymers and proanthocyanidin are found in a wide variety of plants, especially those having a woody habit of growth (e.g., Croton spp. and Calophyllum spp.). A number of different Croton tree species, including Croton sakutaris, Croton gossypifolius, Croton palanostima, Croton lechleri, Croton erythrochilus and Croton draconoides, which are endemic to South America, produce a red viscous latex sap called Sangre de Drago or “Dragon's Blood”. The red viscous latex is known for its medicinal properties. For example, U.S. Pat. No. 5,211,944 describes the isolation of an aqueous soluble proanthocyanidin polymer composition from Croton spp. (See also, Ubillas et al., 1994, Phytomedicine, 1:77). The isolation of an aqueous soluble proanthocyanidin polymer composition from Calophyllum inophylum in U.S. Pat. No. 5,211,944.

In an embodiment, a proanthocyanidin polymer from C. lechleri, or a composition thereof, is crofelemer. Crofelemer (CAS 148465-45-6) is an oligomeric proanthocyanidin of varying chain lengths derived from the Dragon's Blood of Croton lechleri, a tree of the family Euphorbiaceae, which is sustainably harvested under fair trade work practices in the Amazon. It has an average molecular weight of approximately 1900 Da to approximately 2700 Da. The monomers comprising crofelemer comprise catechin, epicatechin, gallocatechin, and epigallocatechin. The chain length of crofelemer ranges from about 3 to about 30 units with an average chain length of about 7 or 8 units. Crofelemer has the chemical formula: (C_{15·n}H_{12·n+2}O_{6.5·n}, wherein n=number of unit) and a molecular mass of 860-9100 g/mol (approximately 2500 Da). The antisecretory mechanism of action of crofelemer involves the targeting and inhibition of two, distinct intestinal chloride channels, namely, the cystic fibrosis transmembrane regulator conductance (CFTR) channel, which is a cAMP-stimulated Cl⁻ channel, and the calcium-activated chloride channel (CaCC), as reported, for example, by Tradtrantip, L. et al., 2010, “Crofelemer, an Antisecretory Antidiarrheal Proanthocyanidin Oligomer Extracted from Croton lechleri, Targets Two Distinct Intestinal Chloride Channels”, Mol. Pharmacol., 77(1):69-78). A general structure of crofelemer is shown below. In the structure, an H at the R position of the structure signifies procyanidin; an OH at the R position of the structure signifies prodelphinidin.

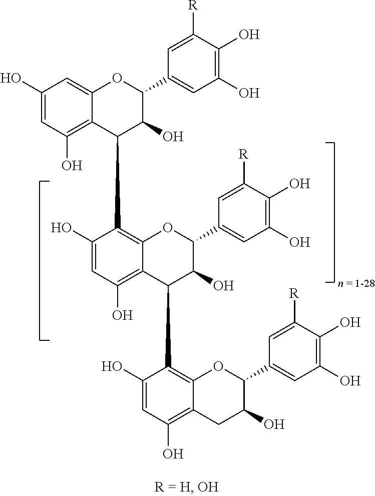

In accordance with an embodiment of the invention, crofelemer, or a pharmaceutically acceptable formulation or composition comprising crofelemer, is employed in the treatment methods as the proanthocyanidin polymer from Croton lechleri.

In an embodiment, SP 303, an oligomeric proanthocyanidin from Croton lechleri, (also known as crofelemer) is the proanthocyanidin polymer from Croton lechleri, or a pharmaceutically acceptable formulation or composition comprising SP 303, which is suitable for use in the treatment methods of the invention. SP-303 (R. Ubillas et al., 1994, Phytomedicine, 1:77-106) is largely composed of purified proanthocyanidin oligomers (−)-epigallocatechin, (+)-gallocatechin, (−)-epicatechin and (+)-catechin and is suitable for use in the enteric and non-enteric formulations and compositions for administration in the treatment methods described herein.

In another embodiment, SB 300, a proanthocyanidin polymer extract from Croton lechleri is the proanthocyanidin polymer from Croton lechleri, or a pharmaceutically acceptable formulation or composition comprising SB 300, which is suitable for use in the treatment methods of the invention. SB 300, as described, for example, by Fischer, H. et al., (2004, J. Ethnopharmacol., 93(2-3):351-357) provides a natural product extract that is particularly amenable for both enteric and non-enteric formulations and compositions, and is highly functional and cost-effective in the treatment methods described herein.

A pharmaceutically acceptable composition comprising a proanthocyanidin polymer from Croton lechleri and employed in the treatment methods of the invention can be obtained from C. lechleri, e.g., as described in WO 00/47062 to Shaman Pharmaceuticals, Inc., the contents of which are incorporated herein, and formulated as a food or dietary supplement or nutraceutical formulation.

In other embodiments, compositions useful in the methods of the invention comprise a raw latex obtained from a Croton species or a Calophyllum species, or an extract obtained from a Croton species or a Calophyllum species, which are not specifically polymeric proanthocyanidin polymer compositions. Exemplary extracts are described in Persinos et al., 1979, J. Pharma. Sci., 68:124 and Sethi, 1977, Canadian J. Pharm. Sci., 12:7.

In an embodiment, the proanthocyanidin polymer from Croton lechleri is formulated with an enteric coating or matrix in a variety of dosage formats known in the art (See, e.g., WO 00/47062 and U.S. Pat. Nos. 7,441,744 and 7,323,195, the contents of which are incorporated herein, and as briefly described below. In another embodiment, the proanthocyanidin polymer is formulation without an enteric coating or matrix. Both enteric and non-enteric forms of the proanthocyanidin polymer from Croton lechleri, for example, SB 300 or SP 303, are intended for use in the methods of the present invention.

### Preparation of Proanthocyanidin Polymer Compositions and Formulations

The proanthocyanidin polymer composition, effective for treating secretory diarrhea according to the invention, is comprised of monomeric units of leucoanthocyanidins. More particularly, the composition is comprised of proanthocyanidin polymers of 2 to 30 flavonoid units, preferably 2 to 15 flavonoid units, more preferably 2 to 11 flavonoid units and most preferably an average of 7 to 8 flavonoid units with a number average molecular weight of approximately 2500 Da. The proanthocyanidin polymer composition is preferably soluble in an aqueous solution. Preferred for use in the methods according to the invention is a proanthocyanidin polymer from C. lechleri; such a C. lechleri proanthocyanidin polymer may be in the form of a pharmaceutically acceptable composition.

Examples of proanthocyanidin polymeric compositions useful in the present invention are preferably isolated or purified from a Croton spp., namely, Croton lechleri, or Calophyllum spp. by any method known in the art. For example, the proanthocyanidin polymer composition may be isolated from a Croton spp. or Calophylum spp. by the method disclosed in U.S. Pat. No. 5,211,944 or in Ubillas et al. (1994, Phytomedicine, 1:77-106, called SP 303 therein), both of which are incorporated herein by reference. Other isolation methods are described in U.S. Pat. Nos. 7,556,831 and 8,067,041 (Example 2), the contents of which are incorporated herein. PCT application PCT/US00/02687, published as WO 00/47062, the contents of which are incorporated herein, also discloses a method of manufacturing a proanthocyanidin polymeric composition isolated from Croton spp. or Calophyllum spp. and enteric formulations of proanthocyanidin polymer dietary supplements, as well as methods of their preparation. Another illustrative method for isolating proanthocyanidin polymer from C. lechleri (such as crofelemer) is found in U.S. Pat. Nos. 7,341,744 and 7,323,195, the contents of which are expressly incorporated herein. As described above, the SP 303 and SB 300 purified forms of oligomeric proanthocyanidin polymer from Croton lechleri are suitable for use in the treatment methods of the invention.

In an embodiment, the proanthocyanidin polymer composition may be generally isolated by the following process, such as provided in U.S. Pat. No. 7,341,744, the contents of which are incorporated herein. Latex collected from Croton lechleri plants is mixed with purified water (preferably one part latex to two parts purified water). Any insoluble material in the latex solution is allowed to settle, e.g., by leaving the mixture at 4° C. overnight (12 hours). The supernatant is pumped away from the residue and is extracted with a short chain alcohol, such as n-butanol. The extraction is preferably performed multiple times, such as three times. After each extraction, the alcohol phase is discarded and the aqueous phase is retained. The aqueous phase is concentrated, for example, using an ultrafiltration device with a 1 kD cut-off membrane. This membrane can be a low protein binding cellulose membrane, or, alternatively, a polypropylene, teflon or nylon membrane can be used. The membrane used should be compatible with acetone. The purpose of the ultrafiltration is to remove the water from the material.

The retentate from the ultrafiltration is then concentrated to dryness, for example using tray-dryers at approximately 37° C. (±2° C.). The dried material is subsequently dissolved in water and is then chromatographed on a cation exchange column (e.g., a CM-Sepharose column) and a size exclusion column (e.g., an LH-20 column). In the preferred two column system, material is run over a CM-Sepharose and then an LH-20 column in a series. Specifically, the dissolved material is loaded onto the cation exchange column and is then washed with purified water. The proanthocyanidin polymer material is eluted from the cation exchange column with an aqueous acetone solution (preferably 30% acetone), thereby loading the proanthocyanidin polymer material onto the sizing column. The sizing column is disconnected from the cation exchange column and the material is then eluted off of the sizing column with an aqueous acetone solution (preferably 45% acetone). The fractions are collected and monitored with a UV detector, e.g., at a wavelength of 460 nm. Fractions containing the proanthocyanidin polymer material are combined and concentrated, for example, by ultrafiltration using, e.g., a 1 kD cut-off membrane (as described above for the ultrafiltration step prior to the chromatography steps). The retentate may then be concentrated to dryness using a suitable drying method, such as, but not limited to, a rotary evaporator, at a temperature of approximately 37° C. (±2° C.). Other suitable drying methodologies include, but are not limited to, tray drying and spray drying. Example 10 of U.S. Pat. No. 7,341,744 provides additional, non-limiting, methodology for preparing a composition comprising proanthocyanidin polymer, which can be used according to the invention. A detailed protocol for isolating an enriched proanthocyanidin polymer extract suitable for use in the methods of the invention is described in WO 00/47062 as noted herein above.

### Methods of Treatment and Applications of Use

The invention is directed to methods of treating diarrhea, especially watery diarrhea resulting from various pathogenic and non-pathogenic causes, for example, illness or disease, pancreatitis, environmental conditions, bacterial, parasitic, protozoan, viral, infection, helminth or worm infection, dietary indiscretions, CID, etc., in young and adult companion animals. The methods involve administering to a companion animal in need of such treatment, a pharmaceutically acceptable composition comprising a proanthocyanidin polymer from a Croton species or Calophyllum species in an amount effective to treat the diarrhea. In preferred embodiments, the proanthocyanidin polymer is from a Croton species, namely, Croton lechleri. Treating the diarrhea can involve reducing the severity and duration of the diarrhea in the animal. Treating the diarrhea can also involve increasing the survivability of the animal undergoing treatment, for example, by reducing or eliminating dehydration in the animal. In an embodiment, the diarrhea is secretory or watery diarrhea. Treating the diarrheal-afflicted animals with the proanthocyanidin polymer from Croton lechleri results in improving overall health of the animals so that their natural immune systems can function in overall health maintenance and in warding off other, or secondary problems, thereby reducing morbidity and mortality.

In addition, the methods of the invention further include methods of preventing or reducing the incidence or severity of diarrhea in young and adult companion animals having been exposed to or suspected of having been exposed to conditions or pathogens that may result in diarrhea. For example, when a companion animal is known to have or is suspected of having come into contact with another animal or pet with diarrhea or a diarrhea-causing illness or infection, the companion animal that has or could have come into contact with an ill or infected animal, or its feces or bodily fluids, may be administered a C. lechleri proanthocyanidin polymer composition of the invention to prevent diarrheal disease or at least reduce the incidence or severity of the disease or the symptoms thereof.

In an embodiment of the invention, the methods are directed to treating non-human companion animals, including adult animals and young animals, that have watery diarrhea or CID, that have or are suspected of having been in contact with one or more other animals posing a risk of causing a diarrhea condition in the companion animal. In embodiments, the methods are directed to treating a companion animal or pet weighing, without limitation, up to 40 kg, 50 kg, or 60 kg, or weighing, on average, 20 kg.

According to the methods of the invention, young and adult companion animals can be treated with a proanthocyanidin polymer from C. lechleri, e.g., SB 300, with crofelemer (e.g., SP 303), or a botanical extract derived from C. lechleri, for one, two, three, four, five, six, seven, eight, nine, or ten days, etc. The C. lechleri proanthocyanidin polymer can be administered to the animal on consecutive days or intermittently, such as every other day, every two days, every three days, every four days, and the like. In an embodiment, the C. lechleri proanthocyanidin polymer is administered to a companion animal for two or three consecutive days in one or two doses per day. The present methods provide treatment of a companion animal having, or at risk of having, diarrhea, particularly, watery diarrhea, with a proanthocyanidin polymer from C. lechleri, or composition thereof, providing for an adequate amount and appropriate distribution of the proanthocyanidin polymer in the gut of the animal so as to treat the diarrhea and/or its symptoms and optimally cure the diarrhea in the animal.

In an embodiment of the diarrhea treatment methods of the invention, a solid oral dosage form, including a chewable formulation, especially a soft, chewable formulation, of a proanthocyanidin polymer from C. lechleri as described herein is orally administered to a companion animal in need thereof.

In an embodiment of the diarrhea treatment methods of the invention, a reconstituted suspension of a formulation of proanthocyanidin polymer from C. lechleri is orally administered to animals, young or adult, (or those exposed or suspected to have been exposed to conditions or agents causing diarrhea) in a volume of from 1 to 50 mL, such as 10 mL. By way of example, a 10 mL volume of an oral suspension containing 40 mg of C. lechleri proanthocyanidin polymer may be administered to a 20 kg dog at a dose of 2 mg/kg twice per day for three days (or six treatment doses).

In a particular embodiment, a formulation or composition comprising a botanical extract derived from C. lechleri, SB 300, or SP 303, may be provided in a dosage form, e.g., a gel, paste, or gel-paste; or a chewable, e.g., a hard or soft chewable, solid dosage form, or suspension formulation that is orally administered to a young or adult companion animal twice daily for three days, preferably, three consecutive days. In a particular embodiment, the formulation comprises beads (nano or microparticles) comprising enterically coated SB 300 or SP 303 and is orally administered to the animal. The dosage form, in specific embodiments, contains 20 to 50 mg of the active ingredient, particularly 40 mg. The methods and formulations are especially suitable for the normalization of stool formation in a short time period, e.g., less than a week or less than two weeks; for mitigation of weight loss; and reduction in supportive care costs, rehydration therapies, such as oral rehydration, in a treated companion animal afflicted with diarrhea.

In accordance with the described methods, the C. lechleri proanthocyanidin polymer composition reduces chloride flux across intestinal epithelial cells and reduces fluid movement into the intestinal lumen, which results in fluid loss and dehydration associated with secretory diarrhea. Therefore, the pharmaceutically acceptable formulations and methods of the invention are useful in both prophylactic and therapeutic applications in the treatment of secretory/watery diarrhea from various causes, especially in preventing the symptoms of dehydration and electrolyte loss that accompany diarrhea, e.g., secretory/watery diarrhea.

In an embodiment, young and adult companion animals may be treated prophylactically with a C. lechleri proanthocyanidin polymer composition, such as SB 300 or SP 303, in enteric or non-enteric form, to prevent or reduce the risk or severity of the debilitating effects of watery diarrhea and its associated symptoms, e.g., dehydration and weight loss. According to the treatment method, a C. lechleri proanthocyanidin polymer composition is administered to young and adult companion animals at a suitable time to protect the animals from diarrhea outbreaks typically caused by infection, disease and/or adverse environmental conditions. Also according to the treatment method, a C. lechleri proanthocyanidin polymer composition, e.g., a solid oral dosage form, including a chewable form, is administered to young and adult companion animals prophylactically, at a suitable time prior to receiving a cancer treatment, e.g., chemotherapy, to protect the animals from diarrhea outbreaks typically associated with, or as a consequence of, such cancer treatment. Administering a C. lechleri proanthocyanidin polymer composition to companion animals can also serve to ameliorate or reduce the risk of the animals' suffering from a more serious or severe form of diarrhea relative to animals that are not provided with the C. lechleri proanthocyanidin polymer composition prior to or during an outbreak of disease or infection or prior to succumbing to CID. In embodiments, the C. lechleri proanthocyanidin polymer composition can be provided as an enteric or a non-enteric formulation and can be, for example, SB 300 or SP 303.

The dose and regimen of C. lechleri proanthocyanidin polymer composition administration are within the skill of the practitioner to determine and will depend on the environmental conditions and health of the animals to be treated. The animals can be prophylactically treated a with C. lechleri proanthocyanidin polymer composition according to the invention, for example and without limitation, for one to seven days, one to six days, one to four days, one to three days, or one or two days prior to onset of symptoms or disease. The treatment regimen can involve one, two, three, four, five, six, seven or more days, of C. lechleri proanthocyanidin polymer composition administration to the animals, modified or adjusted as necessary or desired, once or multiple times, e.g., twice, three or four times, per day. The animals can be regularly observed and monitored for health improvements and weight gain.

### Physiologically and Pharmaceutically Acceptable Formulations

The C. lechleri proanthocyanidin polymer composition can be provided in any physiologically, pharmaceutically, or therapeutically acceptable form. The pharmaceutically acceptable composition can be formulated for oral administration as, illustratively, but without limitation, powders; crystals; granules; small particles, including particles sized on the order of micrometers, e.g., microspheres and microcapsules; particles sized on the order of millimeters, particles sized on the order of nanometers, e.g., nanoparticles; beads; microbeads; pellets; pills; tablets; microtablets; compressed tablets or tablet triturates; molded tablets or tablet triturates; and in capsules, which are either hard or soft and contain the composition as a powder, particle, bead, solution or suspension. As noted above, such tablets, pills, capsules, soft chewable forms, and the like may contain a flavor additive for palatability and/or ease in oral administration to the animal in need. The pharmaceutically acceptable composition can also be formulated for oral administration as a solution or suspension in an aqueous liquid, as a liquid incorporated into a gel capsule, as a gel, as a paste or gel paste, as a chewable composition, formulation, or product, or as any other convenient formulation for administration. The composition can be a soft chewable form that is flavored. The composition can be formulated for rectal administration, as a suppository, enema or other convenient form. The proanthocyanidin polymeric composition can also be provided as a controlled release system (See, e.g., Langer, 1990, Science 249: 1527-1533). The composition can be formulated as a dietary supplement or food supplement, e.g., as described in WO 00/47062, for administration to an animal in need thereof according to the present invention.

The pharmaceutically acceptable formulation can also include any type of pharmaceutically acceptable excipients, additives, carriers, or vehicles. As would be understood by one skilled in the art, such pharmaceutically acceptable ingredients, excipients, additives, carriers, or vehicles may be combined to result in an administrable form or dosage form mentioned above. By way of nonlimiting example, diluents or fillers, such as dextrates, dicalcium phosphate, calcium sulfate, lactose, cellulose, kaolin, mannitol, sodium chloride, dry starch, sorbitol, sucrose, inositol, powdered sugar, bentonite, microcrystalline cellulose, or hydroxypropylmethylcellulose can be added to the proanthocyanidin polymer composition to increase the bulk of the composition. In addition, binders, such as, but not limited to, starch, gelatin, sucrose, glucose, dextrose, molasses, lactose, acacia gum, sodium alginate, extract of Irish moss, panwar gum, ghatti gum, mucilage of isapgol husks, carboxymethylcellulose, methylcellulose, polyvinylpyrrolidone, Veegum and starch arabogalactan, polyethylene glycol, ethylcellulose, and waxes, can be added to the formulation to increase its cohesive qualities. Further, lubricants, such as, but not limited to, talc, magnesium stearate, calcium stearate, stearic acid, hydrogenated vegetable oils, polyethylene glycol, sodium benzoate, sodium acetate, sodium chloride, leucine, carbowax, sodium lauryl sulfate and magnesium lauryl sulfate can be added to the formulation. Also, glidants, such as, but not limited to, colloidal silicon dioxide or talc can be added to improve the flow characteristics of a powdered formulation. Disintegrants, such as, but not limited to, starches, clays, celluloses, algins, gums, crosslinked polymers (e.g., croscarmelose, crospovidone, and sodium starch glycolate), Veegum, methylcellulose, agar, bentonite, cellulose and wood products, natural sponge, cation-exchange resins, alginic acid, guar gum, citrus pulp, carboxymethylcellulose, or sodium lauryl sulfate with starch can also be added to facilitate disintegration of the formulation in the intestine.

In some embodiments, the pharmaceutically acceptable formulations contain the proanthocyanidin polymer composition with an enteric coating, in addition to another pharmaceutically acceptable vehicle. In an embodiment, the proanthocyanidin polymer composition can be directly-compressed into a tablet or pill. The tablet or pill can be without excipients and of pharmaceutically acceptable hardness and friability, optionally, with a lubricant, e.g., without limitation, magnesium stearate, and enteric coated. In another embodiment, the pharmaceutically acceptable compositions containing the proanthocyanidin polymer composition alternatively include one or more substances that either neutralize stomach acid and/or enzymes or are active to prevent secretion of stomach acid. These formulations can be prepared by methods known in the art (See, e.g., methods described in Remington's “The Science and Practice of Pharmacy,” 22nd Edition, Editor-in-Chief: Lloyd V Allen, Jr., Pharmaceutically acceptable Press, Royal Pharmaceutically acceptable Society, London, U K, 2013; and U.S. Pat. No. 7,323,195).

In an embodiment, the proanthocyanidin polymer composition is formulated with a substance that protects the proanthocyanidin polymer and/or the polymer composition from the stomach environment. For such protection, the proanthocyanidin polymer composition can be enteric coated. Enteric coatings include those coatings that remain intact in the stomach, but will dissolve and release the contents of the dosage form once it reaches the small intestine. A large number of enteric coatings are prepared with ingredients that have acidic groups such that, at the very low pH present in the stomach, i.e. pH 1.5 to 2.5, the acidic groups are not ionized and the coating remains in an undissociated, insoluble form. At higher pH levels, such as in the environment of the intestine, the enteric coating is converted to an ionized form, which can be dissolved to release the proanthocyanidin polymer composition. Other enteric coatings remain intact until they are degraded by enzymes in the small intestine, and others break apart after a defined exposure to moisture, such that the coatings remain intact until after passage into the small intestines. A variety of polymers are useful for the preparation of enteric coatings, and the application of an enteric coating to the proanthocyanidin polymer composition can be accomplished by any method known in the art for applying enteric coatings, as may be found, for example, and without limitation, in U.S. Pat. Nos. 7,323,195 and 7,341,744, incorporated herein by reference.

In another embodiment, the pharmaceutically acceptable composition of the proanthocyanidin polymer composition is formulated as enteric coated granules or powder (microspheres with a diameter of 300-500 microns) provided in either hard shell gelatin capsules or suspended in an oral solution for pediatric administration. The enteric coated proanthocyanidin polymer composition powder or granules can also be mixed with food or drink, particularly for administration to young or particularly ill animals. Such preparations may be prepared using techniques well known in the art. In addition, the proanthocyanidin polymer composition granules and powder can be prepared using any method known in the art, such as, but not limited to, crystallization, spray-drying or any method of comminution, preferably using a high speed mixer/granulator, as described, for example and without limitation, in U.S. Pat. No. 7,323,195, incorporated herein by reference.

In other embodiments, the proanthocyanidin polymer composition is in the form of an aqueous suspension in admixture with suitable excipients. Non-limiting examples of excipients that are suitable for the manufacture of aqueous suspension include suspending agents, for example, methylcellulose, sodium carboxymethylcellulose, hydroxypropylmethylcellulose, sodium alginate, polyvinyl-pyrrolidone, gum tragacanth and gum acacia; dispersing or wetting agents, which may be a naturally-occurring phosphatide, e.g., lecithin, or condensation products of an alkylene oxide with fatty acids, e.g., polyoxyethylene stearate, or condensation products of ethylene oxide with long chain aliphatic alcohols, e.g., heptadecaethyleneoxycetanol, or condensation products of ethylene oxide with partial esters derived from fatty acids and a hexitol, for example, polyoxyethylene sorbitol monooleate, or condensation products of ethylene oxide with partial esters derived from fatty acids and hexitol anhydrides, such as polyethylene sorbitan monooleate. The aqueous suspensions may also contain one or more preservatives, for example ethyl, or n-propyl, p-hydroxybenzoate, one or more coloring agents, one or more flavoring agents, and one or more sweetening agents, e.g., sucrose, saccharin or aspartame.

Dispersible powders and granules suitable for the preparation of an aqueous suspension by the addition of water provide the proanthocyanidin polymer composition in admixture with a dispersing or wetting agent, suspending agent and one or more preservatives. Suitable dispersing or wetting agents and suspending agents are exemplified by those stated above. Additional excipients, for example, sweetening, flavoring and coloring agents, may also be present in the dispersible powders, granules and final aqueous suspension.

In an embodiment, the proanthocyanidin polymer composition is a gel or gel formulation. In an embodiment, the proanthocyanidin polymer composition is a paste formulation. In an embodiment, the paste formulation contains a purified botanical extract derived from C. lechleri. In another embodiment, the paste formulation contains enterically coated beads comprising SB 300 or SP 303. In an embodiment, the paste formulation contains enteric protected SB 300 beads. In an embodiment, the gel or paste is contained or preloaded in a delivery device, such as a syringe, e.g., a needle-less syringe, or other type of applicator or delivery system, especially for oral delivery. A gel or paste formulation is particularly suited for administration to young animals, but also is applicable for adult animals, especially those that are very ill. In an embodiment, the gel or paste is not contained in a delivery device, but is administered to the roof of the mouth of the animal, particularly one that is too incapacitated or ill to eat or drink, thereby eschewing an oral or other mode of administration. In an embodiment, the gel comprises pH-sensitive polymeric particles, such as microparticles or nanoparticles, to allow for pH-dependent uptake of the active compound into cells and/or the pH-dependent release of the active compound in different pH environments in an animal. Processes for generating granules and particles comprising the proanthocyanidin polymer composition or a compressible form thereof are as known and practiced in the art, and as provided, for example, in U.S. Pat. No. 7,341,744, the contents of which are incorporated by reference herein. In an embodiment, gels are prepared for oral delivery and contain copolymers, such as poloxamers and Pluronics of different types, e.g., Pluronic F.

In other embodiments, the proanthocyanidin polymer composition is in a paste formulation, preferably for oral administration, or in a solid dosage form, such as a chewable solid dosage form that is soft, preferably for oral administration. By way of example, an oral paste may comprise, without limitation, an oily vehicle or excipient, such as a hydrophobic oily vehicle, a basifying agent, a flavoring agent and a coloring agent. Illustrative and nonlimiting examples of hydrophobic oily vehicles include vegetable oil, triglyceride or polypropylene glycol, as well as a thickening agent, e.g., aluminum stearate. Flavoring agents can include, for example, fruit flavors, mint flavors, honey flavor, beef flavor, fowl flavor, fish flavor, vegetable flavor, and other natural and organic flavorings known to those skilled in the art. Coloring agents can include, for example, iron oxide or titanium dioxide. Alternatively, the oily vehicle can be liquid paraffin or other suitable waxes, including a thickening agent. In an embodiment, the paste or solid formulation contains beads with enterically coated SB 300 or SP 303, which is administered to a companion animal, such as, for example, a dog or a cat, at a dose of 2 mg/kg or at a dose of 4 mg/kg. More particularly, a paste or solid formulation containing enterically coated SB 300 or SP 303 microparticles or beads is administered to the animal at a dose of 2 mg/kg, twice a day for one, two, three, or more days. In an embodiment, the paste or solid form containing enteric protected SB 303 microparticles is administered twice a day to a companion animal in need thereof. In an embodiment, an effective amount of the C. lechleri proanthocyanidin polymer as active ingredient is formulated into a chewable form, such as a tablet, pill, or capsule, that is orally administered and flavored to be palatably acceptable to a companion animal. In an embodiment, microparticles of enteric coated SP 303 are formulated into a soft, chewable, flavored composition or product for oral administration to the animal such as a dog.

In another embodiment, oily suspensions may be formulated by suspending the C. lechleri proanthocyanidin polymer as active ingredient in a vegetable oil, e.g., arachis oil, olive oil, sesame oil or coconut oil, or in mineral oil, such as liquid paraffin. The oily suspensions may contain a thickening agent, e.g., beeswax, hard paraffin or cetyl alcohol. Oral preparations can include sweetening agents as mentioned above and flavoring agents to improve palatability. Pharmaceutically acceptable preservatives, for example, an anti-oxidant such as ascorbic acid, can also be added to such compositions.

The C. lechleri proanthocyanidin polymer pharmaceutical compositions used in the methods of the invention may also be in the form of an oil-in-water emulsions. The oily phase may be a vegetable oil such as olive oil or arachis oil, or a mineral oil such as liquid paraffin or mixtures of these oils. Examples of emulsifying agents include, without limitation, naturally-occurring phosphatides, e.g., soy bean, lecithin, and esters or partial esters derived from fatty acids and hexitol anhydrides, e.g., sorbitan monooleate, and condensation products of partial esters with ethylene oxide, e.g., polyoxyethylene sorbitan monooleate. Sweetening, coloring and flavoring agents can be included in the emulsions.

Syrups and elixirs containing the C. lechleri proanthocyanidin polymer may also be formulated with sweetening agents, for example, glycerol, propylene glycol, sorbitol or sucrose. Such formulations may also contain a demulcent, a preservative and flavoring and coloring agents. The pharmaceutical compositions may be in the form of a sterile, orally deliverable or administrable aqueous or oleagenous suspension. This suspension may be formulated according to methods known in the art using suitable dispersing or wetting agents and suspending agents, such as those mentioned above. The sterile pharmaceutical preparation may also be a sterile solution or suspension in a non-toxic parenterally-acceptable diluent or solvent, for example, a solution in 1,3-butane diol. Illustrative, acceptable vehicles and solvents that may be used in the preparations include water, Ringer's solution and isotonic sodium chloride solution. Co-solvents, e.g., ethanol, propylene glycol or polyethylene glycols, may also be included. In addition, sterile, fixed oils, e.g., any bland, fixed oil such as synthetic mono- or diglycerides, are conventionally employed as solvents or suspending media and may be used. In addition, fatty acids, such as oleic acid and the like, may be used in injectable preparations.

### Dosage Forms and Administration

In an embodiment, for treating watery diarrhea, including CID, in companion animals, for example, without limitation, dogs and cats, the proanthocyanidin polymer composition is in powder, e.g., reconstitutable powder, form. In another embodiment, for treating watery diarrhea, including CID, in companion animals, for example, without limitation, dogs and cats, the proanthocyanidin polymer composition is in a solid dosage form, e.g., a chewable solid form. The composition may be enterically coated or not enterically coated. The powder form of the proanthocyanidin polymer composition used for treatment is reconstituted or mixed with liquid, such as oral electrolytes, milk or a milk replacer, water, physiological saline, to produce a liquid form or suspension. Milk replacer is generally a source of protein from different origins (for example, milk from a different species, soy, or eggs) and energy (lactose and fat) given to young animals to replace milk from the mother. The chewable solid dosage form may be flavored, e.g., beef, chicken or fish flavor, for optimal palatability to the animal being treated.

In an embodiment, the powder form of the proanthocyanidin polymer composition is provided in the form of individual dosages in packets, e.g., packaged dosage forms, wherein some number of individual packets are provided for use in a treatment regimen. In certain embodiments, the total packaged dosage form contains from 0.5 mg to 1000 mg of the proanthocyanidin polymer composition. In other embodiments, the total packaged dosage from contains from 1 mg to 100 mg, 5 mg to 500 mg, 10 mg to 50 mg, 50 mg to 200 mg, 100 to 300 mg, 100 to 250 mg, or 250 mg of the proanthocyanidin polymer composition. The number of individual doses that can be packaged and provided together is not intended to be limiting, and can include, for example, one to twenty packaged doses; one to ten packaged doses; two, four, six, eight, ten, twelve, or more packaged doses, as well as numbers of packaged doses in-between the foregoing, for efficiency of use, handling and for commercial efficacy. Those skilled in the art will appreciate that due to the higher purity of compositions such as SP-303 or crofelemer and SB-300, more by weight of SB-300 than SP-303 will need to be used in formulations to achieve the same amount of the active ingredient of the proanthocyanidin polymer composition. SB-300 generally has about 67% by weight of the proanthocyanidin polymer composition while SP-303 has higher purity, for example 99-100%.

In an embodiment, the C. lechleri proanthocyanidin polymer composition is administered or delivered to a young or adult companion animal afflicted with diarrhea and in need thereof by providing the compound in a chewable solid dosage form. In an embodiment, the proanthocyanidin polymer composition formulated as bolus, i.e., a pill, capsule, tablet, or solid chewable form, is orally administered to the animals afflicted with diarrhea or symptoms thereof directly in the mouth. In some embodiments, the treatment regimen comprises administering a dose of 0.05 mg/kg to 100 mg/kg; or a dose of 0.1 mg/kg to 100 mg/kg, or a dose of 0.1 mg/kg to 10 mg/kg; or a dose of 0.5 mg/kg to 50 mg/kg; or a dose of 0.5 mg/kg to 25 mg/kg; or a dose of 1 mg/kg to 25 mg/kg; or a dose of 2 mg/kg to 10 mg/kg, or a dose of 1 mg/kg to 5 mg/kg; as well as dose increments there between, of a C. lechleri proanthocyanidin polymer product, in consideration of the weight of the companion animal. In a particular embodiment, the treatment regimen comprises administering a dose of 2 mg/kg or 4 mg/kg of the product, e.g., as embraced by one dose per animal in need for a determined time period, for example, one, two, or three times per day, for one, two, three, or more days. The product can be provided to the animals in an enteric coated or a non-enteric coated formulation. The product can be provided to an animal in need thereof in portions of the complete dose, in which the portions are administered one or two or more times per day. Alternatively, the complete dose can be administered to an animal in need thereof one or two or more times per day. In a particular embodiment, the treatment encompasses a total dose of 40 mg given two times a day (20 mg/dose) to an animal weighing approximately 20 kg. For larger animals, those greater than 20 kg and, preferably, less than 40 kg, the dosage is 80 mg given two times per day. In an embodiment, the dosed product is the C. lechleri proanthocyanidin polymer composition, SP 303, in enteric or non-enteric form, e.g., a reconstituted powder form or a solid dosage form, including a chewable solid form, optionally flavored according to the particular taste preference(s) of the animal to be treated, at a dose of approximately 2 mg/kg or at a dose of approximately 4 mg/kg. In an embodiment, the dosed product is the C. lechleri proanthocyanidin polymer composition, SB 300 or SP 303, in an enteric form. In an embodiment, the dosed product is the C. lechleri proanthocyanidin polymer composition, SB 300 or SP 303, in a non-enteric form.

The routes of administration of the C. lechleri proanthocyanidin polymer product to afflicted animals are not intended to be limiting. Illustratively, administration can be via any suitable, convenient or preferred route of administration including oral, buccal, dental, periodontal, via food source (animal feed), nutrition source, or libation source, otic, inhalation, endocervical, intramuscular, subcutaneous, intradermal, intracranial, intralymphatic, intraocular, intraperitoneal, intrapleural, intrathecal, intratracheal, intrauterine, intravascular, intravenous, intravesical, intranasal, ophthalmic, biliary perfusion, cardiac perfusion, spinal, sublingual, topical, transdermal, intravaginal, rectal, ureteral, or urethral. In certain embodiments, oral, buccal, and food and/or drink supplement are particularly suitable routes. In an embodiment, the product is an aqueous formulation and is provided to the animal as a drench or directly from a ready-to-use (RTU) bottle directed to the esophageal cavity so as to more effectively reach the animal's intestine/gut for optimal activity. In a related embodiment, administration can also be by inclusion in the regular or special diet of the animal, such as in a functional food for the animals or companion animals. In a particular embodiment, the product is a chewable, flavored solid form, such as a tablet, pill, capsule, pellet, or other suitable solid, orally administrable form.

Dosage forms can include, without limitation, oral, injectable, transdermal, aerosol including metered aerosol, chewable products or pellets, capsules, capsule containing coated particles, nanoparticles, or pellets, capsule containing delayed release particles, capsule containing extended release particles, concentrates, creams and augmented creams, suppository creams, discs, dressings, elixirs, emulsions, enemas, extended release films or fibers, gases, gels, metered gels, granules, delayed release granules, effervescent granules, implants, inhalants, injectable lipid complexes, injectable liposomes, inserts or devices, extended release inserts, intrauterine devices, jellys, liquids, extended release liquids, lotions, augmented lotions, oils, ointments, augmented ointments, pastes, pastilles, pellets, powders, reconstituted powders, extended release powders, metered powders, solutions, drops, concentrated solutions, gel forming solutions/drops, sponges, sprays, metered sprays, suppositories, suspensions, suspensions/drops, extended release suspensions, syrups, tablets/pills, chewable tablets/pills, tablets/pills containing coated particles (nano or microparticles), delayed release tablets/pills, dispersible tablets/pills, effervescent tablets/pills, extended release tablets/pills, orally disintegrating tablets/pills, tapes, troches/lozenges, or other forms. The dosages can be provided as solid, liquid, or suspension formulations, compositions, pharmaceutically acceptable formulations and compositions, physiologically acceptable formulations and compositions, and can include pharmaceutically and physiologically acceptable carrier, excipients, diluents, or vehicles as known and used in the art.

For oral administration, the C. lechleri proanthocyanidin polymer product, or a composition thereof, is preferably encapsulated and formulated with suitable carriers, and the like, in solid dosage forms. Nonlimiting examples of suitable carriers, excipients, diluents and vehicles include lactose, dextrose, sucrose, sorbitol, mannitol, starches, gum acacia, calcium phosphate, alginates, calcium silicate, microcrystalline cellulose, polyvinylpyrrolidone, cellulose, gelatin, syrup, methyl cellulose, methyl- and propylhydroxybenzoates, talc, magnesium, stearate, water, mineral oil, edible oils, and the like. The formulations can also include lubricating agents, wetting agents, emulsifying and suspending agents, preserving agents, sweetening agents and/or flavoring agents. The compositions can be formulated to provide rapid, sustained, extended, or delayed release of the active ingredient after administration to the animal by employing protocols and methods well known in the art. The formulations can also include compounds or substances that reduce proteolytic degradation and promote absorption such as, for example, surface active agents.

As will be appreciated by those having skill in the art, the specific dose can be calculated according to the approximate body weight, body mass, or body surface area of the animal, or the volume of body space or mass to be occupied. The dose also depends on the particular route of administration selected by the practitioner. Further refinement of the calculations necessary to determine an appropriate dosage for treatment is routinely made by those of ordinary skill in the art, for example, using appropriate assays and analytical procedures, such as has been described for certain compounds (e.g., Howitz et al., Nature, 425:191-196, 2003). Exact dosages can be determined based on standard dose-response studies. Therapeutically effective doses for treatment of afflicted animals can be determined, by titrating the amount of the active product given to the animal to arrive at the desired therapeutic effect, while minimizing side effects.

For use in treating watery diarrhea, or CID and the symptoms thereof in young and adult companion animals in accordance with the methods of the invention, a therapeutically acceptable form of the C. lechleri proanthocyanidin polymer composition is administered, particularly orally administered, in an amount ranging from 0.1 to 100 mg/kg per day, once, twice or more daily. In other embodiments, the amount can range from about 0.1 to about 25 mg/kg/day, once, twice or more daily; or from about 0.1 to about 10 mg/kg/day, once, twice or more daily; or from about 0.1 to about 5 mg/kg/day, once, twice or more daily; or from about 0.1 to about 0.5 mg/kg/day, once, twice or more daily; or from about 0.5 to about 25 mg/kg/day, once, twice or more daily. In other embodiments, the dose can be 0.1 mg/kg, 0.2 mg/kg, 0.3 mg/kg, 0.4 mg/kg, 0.5 mg/kg, 0.6 mg/kg, 0.7 mg/kg, 0.8 mg/kg, 0.9 mg/kg, 1 mg/kg, 2 mg/kg, 3 mg/kg, 4 mg/kg, 5 mg/kg, etc., as well as incremental dose amounts in between. In still other embodiments, the amount can range from about 1 to about 10 mg/kg/day once, twice or more daily; or from about 1 to about 5 mg/kg/day once, twice or more daily, or from about 1 to about 4 mg/kg/day once, twice or more daily; or from about 0.1 to about 4 mg/kg/day once, twice or more daily, or from about 2 mg/kg/day to about 4 mg/kg/day once, twice or more daily. In other embodiments, the foregoing amounts of the C. lechleri proanthocyanidin polymer composition are administered, for example, twice daily, three times daily, four times daily, or more than four times daily, rather than once per day.

In other embodiments, for the treatment methods, a suitable dose for the C. lechleri proanthocyanidin polymer product, or the C. lechleri proanthocyanidin polymer composition, such as SP 303 or SB 300, may range from about 0.01 mg to about 1000 mg, either daily or multiple times per day. In an embodiment, a suitable dose may range from about 0.1 mg to about 100 mg or from 1 mg to about 10 mg, either daily or multiple times per day. In an embodiment, a suitable dose may range from about 10 mg to about 350 mg, either daily or multiple times per day. In an embodiment, a suitable dose may range from about 20 mg to about 400 mg, either daily or multiple times per day. In an embodiment, a suitable dose may range from about 50 mg to about 100 mg, either daily or multiple times per day; from about 100 mg to about 250 mg, either daily or multiple times per day. In an embodiment, a suitable dose may range from about 40 mg to about 300 mg, either daily or multiple times per day. It will be understood that the ranges include the lower and higher amounts specified, as well as amounts in between. The doses administered multiple times per day can be given for consecutive days, e.g., two days, three days, four days, five days, six, days, seven days, or more, in some embodiments. A dose administered multiple times per day may embrace two, three, four, five, six, or more times per day. Other dosing schedules, such as every other day, or every third day, every fourth day, etc. are embraced by the invention. In addition, one having skill in the art will appreciate that doses and amounts administered to the animal can vary and may be less or more than the aforementioned, representative ranges and amounts, given the wide range of weights of companion animals undergoing treatment, as well as the differences among animal species. In an embodiment the C. lechleri proanthocyanidin polymer is SB 300 or SP 303. In an embodiment the C. lechleri proanthocyanidin polymer is enterically coated SB 300 or SP 303. In an embodiment the C. lechleri proanthocyanidin polymer is non-enterically coated SB 300 or SP 303.

In some embodiments, daily doses, including multiple daily doses, e.g., twice or three times a day, of the C. lechleri proanthocyanidin polymer product may be 0.1 mg, 0.5 mg, 1 mg, 2 mg, 4 mg, 5 mg, 10 mg, 15 mg, 20 mg, 25 mg, 40 mg, 50 mg, 80 mg, 100 mg, 150 mg, 175 mg, 200 mg, 225 mg, 250 mg, 275 mg, 300 mg, 325 mg, 350 mg, 375 mg, 400 mg, 500 mg (or there between) per animal. Close approximations of these doses are also encompassed in the methods. Administration schedules may also be altered to achieve a therapeutically effective concentration of the C. lechleri proanthocyanidin polymer to treat the diarrhea and its symptoms as described herein. By way of specific, yet nonlimiting example, a suitable dosage amount for use in the methods according to the invention is 2 mg/kg administered twice daily or 4 mg/kg/dose administered twice daily. In some embodiments, the compound may be administered once per day, twice per day, thrice per day, 4 times per day, 5 times per day, 7 times per day or 10 times per day. Often the dosage is divided into equal parts administered throughout the day, however in some embodiments related to treating more severe or entrenched symptoms, it may be useful to tailor the dosage administration schedule so that most of the daily treatment is administered at a predetermined time of the day, e.g., the beginning half of the day. By way of example, a first dose may be administered to the animal, and then a second (or subsequent) dose(s) may be administered after a given number of hours, such as after 1 to 3 hours, 1 to 6 hours, 1 to 12 hours, or there between. In some embodiments, about 50% 60%, 70% or 80% of the dosage is administered in the first half of the day. In other embodiments, it may be more appropriate to administer most of the dosage in the latter half of the day so that about 50%, 60%, 70% or 80% of the dosage is administered in the latter half of the day.

It will be understood that the dose amount actually administered can be determined by the practitioner, in the light of the relevant circumstances, including the severity of the disease, condition, or symptoms thereof being treated, the form of the product to be administered, the age, weight, and response of the individual animal receiving treatment, as well as the chosen route of administration.

The methods of the invention further embrace the administration of pharmaceutically acceptable formulations of the proanthocyanidin polymer composition either alone or in combination with other supplements or agents for treatment or amelioration of the symptoms of secretory diarrhea, such as rehydration agents, electrolytes (e.g., sodium, potassium, magnesium, chloride and formulations thereof), antibiotics, gut-lining protectants, such as kaolin, pectin, or bismuth liquid, and fluid adsorbents, such as attapulgite. Other agents may include anti-motility agents. Natural biological products, e.g., Lactobacillus or Streptococcus faecium, or probiotics, may also be employed as additives to restore the natural balance of intestinal flora in affected young or adult companion animals.

### EXAMPLES

### Example 1

Evaluation of Oral Administration of a Croton lechleri Proanthocyanidin Polymer Composition, Crofelemer (SP 303), in Dogs

Described in this Example is a multicenter proof-of-concept study of dogs having acute watery diarrhea. The study is conducted in veterinary hospitals to provide a controlled environment. The study is a double-blind, block-randomized format to compare five distinct treatment groups based on the cause of diarrhea, with each of five related placebo groups, as well as a global analysis. The study involves the enrollment of dogs presenting with general acute watery diarrhea for less than three days. A thorough clinical examination of each dog is conducted to determine if the cause of diarrhea is chemotherapy, bacterial infection, pancreatitis, dietary indiscretion or Giardia infection. Each of these five causes is considered a subgroup, and dogs with any other cause of diarrhea are excluded from the study.

Enrolled dogs are hospitalized at a clinic for four days and treated according to their weight classification with 2-4 mg/kg of enteric-coated micro-granules comprising C. lechleri proanthocyanidin polymer composition (crofelemer); (dosage range according to the size and weight variability of dog species), or an enteric-coated placebo, twice a day for three days. In addition, all enrolled dogs are treated according to the “standard of care” (SOC) for diarrhea, which includes oral or intravenous fluids for rehydration and disease-specific medications such as fenbendazole for Giardia infection, pain control for pancreatitis and an anti-emetic for vomiting (Maropitant citrate). The endpoints being evaluated are feces consistency, a comprehensive gastrointestinal score, duration of diarrhea, frequency of defecation, appetite, attitude and body temperature. Dogs are examined twice a day for four days and assessed using two validated scoring systems: the Nestle Purina Fecal and the Waltham Fecal Scoring systems. Feces samples are taken once a day to establish or confirm the cause of diarrhea and to measure the dry matter content of the stool. Blood chemistry analyses are also studied. The design provides for the evaluation of data by subgroup, and as a whole, across a range of relevant clinical endpoints. As a result, the study may allow confirmation of the clinical benefits for all or some of these subgroups of general acute watery diarrhea.

For the proof-of-concept study, SP 303 is provided in test article packets. Each test article packet is labeled with the test animal number and the sequence of twelve packets allotted per animal enrolled in the trial study, i.e., Dog 1, Packet 1 of 12, etc. Each dog receives twelve packets during the course of the study, since each study dog receives six total treatments. Each packet contains 250 mg of Test Article (SP 303) and is sufficient to treat a 20 kg dog for one treatment. Therefore, a total of two packets are required, per treatment, for treating a dog weighing up to 40 kg. The dose for treatment is 2 mg/kg, twice per day, for six treatments (three days). Each packet of 250 mg of Test Article contains 40 mg of active or placebo ingredient, and is reconstituted into a suspension by adding 10 mL of tap water to produce a suspension of 4 mg/mL of active ingredient. Therefore, for a 20 kg dog to receive the test dose, (20 kg×2 mg/kg×4 mg/mL), the dog is dosed with 10 mL of the suspension.

### Example 2

Evaluation of the Clinical Efficacy of Oral Administration of a Croton lechleri Proanthocyanidin Polymer Composition, Crofelemer (SP 303), for the Treatment of Diarrhea in Dogs

This Example is directed to a blinded, randomized controlled study conducted by veterinarians using canine animals obtained through rescue organizations, shelters and client owners to evaluate the clinical efficacy of oral administration of a C. lechleri proanthocyanidin polymer composition product, namely, crofelemer SP 303, in treating diarrhea. Investigators are blinded to the treatment assignment until the conclusion of the study. A minimum of 60 dogs per trial site are recruited over a 4 month period from different veterinary clinics from the same geographic area. Rescue organizations, shelters and owners are offered a financial incentive for enrollment of their animals in the trial. Consent is given for animals to be confined at the study site for a maximum period of 6 days. During the first 24 hour period of confinement, diarrhea severity is assessed through veterinarian or assistant examination, observation and owner input. If, at the end of the first 24 hours of confinement, the animal is determined to have a baseline fecal score of 6 or greater, the animal is enrolled in the trial, randomized and confined for a period of 5 days with treatment commencing at the beginning of the second 24 hours of confinement.

A study goal is to confirm the clinical efficacy of the crofelemer product SP 303 administered orally in alleviating the clinical signs of diarrhea in dogs. This study also provides data to support pivotal efficacy studies for assessing the crofelemer product in the treatment of chemotherapy induced diarrhea (CID), as well as the more general acute diarrhea/watery diarrhea indications in dogs.

A block randomization with a block size of 6 is utilized in the study. The primary clinical endpoints that are assessed include fecal consistency and duration of diarrhea; the secondary endpoints include safety, attitude/activity of the treated dogs. Written Consent is obtained for the enrollment of all dogs in the study. The dogs suitable for the study include males and females of any breed between the ages of 4 months and 12 years and having a weight between 2 kg and 50 kg. Only dogs that present with diarrhea and have a fecal score of 6 or greater (i.e., watery, liquid stools with little particulate matter) are admitted in the study. All dogs admitted into the study are current on parvovirus vaccinations and are examined thoroughly to determine final inclusion. Dogs should have had diarrhea for 3 days or less to be eligible.

Exclusion criteria: Dogs that have other medical conditions or that are being treated for other medical conditions, as well as dogs with a fecal sore of 4 or below are not admitted in the study. Dogs with bloody diarrhea and a suspicion of parvovirus are excluded. Dogs that have been treated with any prohibited medications within 7 days of the owner's signing informed consent are not enrolled in the study.

Final inclusion: Only dogs that diagnosed with Giardia, acute bacterial intestinal infection, CID, pancreatitis and dietary indiscretion are retained in the study and can complete treatment. Each dog is placed into one of five subgroups. All subgroups are analyzed separately and all subgroups are pooled together as a group for separate analysis. Prohibited medications include Metronidazole, Albendazole, Forti Flora, Centrine and Sulfadimethoxine. Treatment with rescue medications is allowed, pursuant to the discretion of the principal investigator (PI).

Test articles and dosage: Two test articles are supplied to clinicians/PIs. The articles are packaged individually for each dog in the study, with only a code number provided by the sponsor on each package. One test article is SP 303 (crofelemer) in a dosage of 2 mg/kg. A second test article is placebo, e.g., saline solution. The final dosage form is enteric coated microgranules of SP 303, to be administered to the dog in a small amount of water: 20 mg of the active ingredient (SP 303) per 1 mL of water, directly in the mouth, administered by a trained veterinary technician. The dose of the active is based on the weight of the animal. Each dog is treated twice a day for three days—six total doses.

Assessments: The evaluator (veterinarian, assistant, or technician) conducts the following assessments: 1) A fecal score is determined at baseline and on treatment days 1, 2, 3, 4, and 5 of the study according to the feces scoring system presented below:

| Description | Score |
|---|---|
| Hard dry stools | 1 |
| Ideal firm, well-formed stools, do not leave marks when picked up | 2 |
| Well-formed stools with slightly moist surface which leave | 3 |
| marks when picked up, almost sticky to touch | |
| Soft moist beginning to loose form | 4 |
| Very soft moist, amorphous | 5 |
| Viscous liquid with some particulate matter | 6 |
| Watery, liquid stools with little particulate matter | 7 |
| Severe watery diarrhea, no particulate matter visible | 8 |
| Hemorrhagic diarrhea | 9 |

The evaluator also scores the general attitude/activity, dehydration status and appetite at baseline and on days 1-5 according to the parameters listed below:

| | Score |
|---|---|
| Attitude/Activity | |
| Normal | 0 |
| Slightly decreased | 1 |
| Moderately decreased | 2 |
| Severely decreased, animal not responsive | 3 |
| Hydration | |
| Normal | 0 |
| Mild change | 1 |
| Moderate change | 2 |
| Severe change, need IV fluid | 3 |
| Appetite | |
| Normal | 0 |
| Ate most | 1 |
| Ate little | 2 |
| Did not eat | 3 |

The evaluator also takes and reports each dog's body temperature. In addition, the number of episodes of defecation per day is recorded by observing the animal from baseline through the treatment period.

Documentation: For documentation, the appropriate source documents are provided to the study site; the documents are kept in each animal's medical file and completed as the study progresses. Site personnel enter data recorded on source documents in the study baseline using Electronic Data Capture (EDC) software. A clinical study monitor visits the sites to verify that all data in the EDC system matches the source documents for each animal.

Procedure: Diagnostic evaluation for all dogs before entry into the study includes: Medical History: Duration and Clinical signs; Vaccination history; Dietary history; Environment/history: Potential for dietary indiscretion: food, foreign body, garbage, compost, manure; Stressful episodes: boarding, grooming, travel, hospitalization, change in family routine, recent move, etc.; Recent medications; Physical Exam/Clinical evaluation: mucous membrane color, hydration, posture, activity/attitude, body condition, abdominal palpation, rectal examination, observation of animal defecation, i.e., tenesmus, signs of pain, characteristics of feces, etc.; Laboratory test: feces are collected during rectal examination. The presence or appearance of parasites is determined by direct smears and flotation, including zinc sulfate flotation, ova, larvae, Giardia, motile protozoa. Clinical labs drawn: CBC/SMAC and Diarrhea Panel prior to enrollment and at end of treatment period. Diarrhea Panel to screen for Campylobacter coli, Campylobacter jejuni, canine distemper virus, canine corona virus, canine parvovirus 2, Clostridium perfringens alpha toxin, Cryptosporidium spp., Giardia and Salmonella spp. Other tests may be conducted as deemed necessary or appropriate by the veterinarian.

If a dog meets the criteria to be included in the study, the evaluator proceeds with filling the scoring sheet for day 1 and administering the first dose. The evaluator also administers and prescribes, at his/her own discretion, any other medication necessary and records the treatment on the concomitant medication case report form. The evaluator also scores the dog on days 2, 3 and 5. The dogs are treated twice a day for three days (6 total doses) and are observed for five days. For each dog, the study is terminated after five days, regardless of the outcome. The appropriate study termination case report form is completed for each animal.

Withdrawals: Any animal that does not comply with the treatment schedule is withdrawn from the study. Any animal suffering from unrelated problems, e.g., injuries, during the course of treatment is withdrawn from the study. Data recorded up to the time of withdrawal from or completion of the study, whichever is shorter, is used in the data analysis for the study.

Adverse Events: All adverse events are reported on the data collection sheet. The study investigator makes the initial decision on the relatedness of the adverse event to the assigned treatment.

Statistical Methods: The study is a randomized, double-blind, prospective comparison study of the C. lechleri proanthocyanidin polymer SP 303 compared with placebo in dogs with acute diarrhea. The objectives of the statistical analyses are to establish the efficacy and safety of the test product in alleviating the clinical signs of diarrhea in dogs. A study center is defined as a treatment administration site under the control and supervision of the same PI. Data are analyzed for each of the 5 subgroups and for the entire population in the study.

Randomization Methods: A single randomization list is generated using SAS PROC PLAN in order to randomize the study. The randomization list is generated in permuted blocks of size greater than two in order to minimize assignment bias at the study site. The list indicates only the treatment code for the assigned treatment. Code break envelopes for each animal are issued to the study site in order to unblind an animal in the case of emergency. Study centers are pooled for all statistical analyses.

Efficacy Variables: The primary efficacy variables of the study are the veterinarian fecal scores (recorded on a 7 point scale, with 7 being the worst diarrhea symptoms) recorded at baseline and on days 1-5 of dosing. The primary efficacy endpoints are the change from baseline fecal scores at each dosing day (days 1-5). The change from baseline fecal scores is computed as baseline fecal score minus the fecal score during the treatment period, i.e., a positive change from baseline score indicates an improvement in diarrhea symptoms.

Secondary Efficacy Variables: Secondary efficacy variables measured for each animal include attitude/activity; hydration; body temperature; number of defecations per day; stool consistency; and appetite. Animal attitude/activity, hydration, appetite and stool consistency are measured on a 0-3 scale with 3 being the most severe rating. Ratings are made on each animal at baseline and on days 1-5 of the treatment period. Body temperature (in ° C.) is measured at baseline and at days 1-5 of treatment. The secondary efficacy variables are the change from baseline values for each variable at each treatment day (days 1-5).

Safety Variables: Primary safety variables: The primary safety variables are the adverse events observed for each animal during the treatment period. Sample Size Justification: The study is an early phase study of the SP 303/crofelemer compound in the treatment of diarrhea in dogs; it is not statistically sized for hypothesis testing.

Analysis Population: An Intent-to-Treat (ITT) population consists of all subjects that are enrolled in the study and receive at least one dose of the study medication. The analysis of the primary and secondary efficacy endpoints is conducted on the ITT population. All safety analyses are conducted on the ITT population.

Analysis of Baseline and Demographic Characteristics: Baseline and demographic characteristics are summarized for all animals in the safety population. Continuous variables are displayed via summary statistics (mean, median, sample size, standard deviation, minimum and maximum) by treatment group. Categorical variables are summarized via counts and percentages by treatment group.

Analysis of Primary Variables: The following hypothesis is tested at each time point (treatment days 1-5) at a one-sided significance level of α=0.025 relative to the primary variables: Hₐ₀: μ_{T}≤μ_{P} vs. Hₐ₁: μ_{T}>μ_{P}, where μ_{T} is the mean change from baseline fecal scale score for the SP 303 treatment group and μ_{P} is the mean change from baseline fecal score for the placebo group. The hypothesis is tested using an F test for treatment effect from an ANOVA analysis using a linear model containing terms for treatment group and baseline fecal score at each time point. Summary statistics (mean, median, sample size, standard deviation, minimum and maximum) are computed by treatment group at each time point (treatment days 1-5). In addition, adjusted treatment group change from baseline mean fecal scores is computed from the ANOVA model at each time point.

Analysis of Secondary Variables: For each of the secondary variables, an ANOVA analysis using a linear model with terms for treatment group and baseline value of the parameter is conducted at each time point. Summary statistics are calculated by treatment group at each time point. Adjusted treatment group change from baseline means is computed from the ANOVA model at each time point.

Analysis of Safety Variables: For the Safety Analyses, summary statistics are calculated. All adverse events (AEs) are observed for each animal from enrollment until termination of the study. Treatment emergent adverse events are summarized by treatment group. A treatment emergent adverse event is an adverse event with a start date on or after the first treatment date for an animal. If an adverse event has a missing start date, it is considered treatment emergent. Prior to analysis, all adverse events (AEs) are coded using the Veterinary Medical Dictionary for Regulatory Activities (VMedDRA). Based on these coded terms, AEs are summarized using system organ class and preferred terms. All AEs are listed.

Missing Values: For all statistical analyses conducted on the primary and secondary endpoints, a complete case analysis is conducted, i.e., no missing values are inputted. In addition, no formal interim analyses are conducted during this study.

### Example 3

Treatment of Diarrhea in Dogs with C. lechleri Proanthocyandin Polymer Composition (SB 300)

This Example describes a small-scale study in dogs having diarrhea and treated with the C. lechleri proanthocyandin polymer composition SB 300. The blinded, controlled study was conducted in three small animal veterinary clinics in Bogota, Columbia. A goal of the study was to evaluate the clinical efficacy of SB 300 administered orally in alleviating the clinical signs of diarrhea in the treated dogs. The study evaluated 48 dogs that were block randomized into three groups. One group was treated with SB 300 enteric coated tablets at a dosage of approximately 4 mg/kg/dose; a second group was treated with placebo; and a third group was treated with a non-enteric form of SB 300 as a powder at a dosage of approximately 4 mg/kg/dose. The test animals included both male and female dogs of at least 20 kg in body weight. The animals were dosed twice a day for three days (6 doses) and observed for 5 days.

The test articles employed in the study were as follows:

Test article 1: SB 300 purified extract from Croton lechleri; minimum dose approximately 4 mg/kg; dosage form: enteric coated tablets; 1 tablet twice a day.
Test article 2: SB 300 purified extract from Croton lechleri; minimum dose approximately 4 mg/kg; dosage form: non-enteric powder form for top dressing of food, twice a day. As an alternative, the powder was dissolved in a small amount of water and orally administered with a syringe if a dog did not eat.
Test article 3: Placebo (dosage form without the active SB 300 product). To ensure the blinding of the study, 50% of the placebo was formulated as tablets and 50% was formulated as powder, dosed twice a day.

For evaluating the animals suitable for the study and the results of the study treatments, feces consistency was measured using a fecal/stool scoring system from 1 to 9, as shown below.

| Description | Score |
|---|---|
| Hard, dry and crumbly stools (like bullets) | 1 |
| Hard dry stools | 2 |
| Ideal firm, well-formed stools, do not leave marks when picked up | 3 |
| Well-formed stools with slightly moist surface which leave marks | 4 |
| when picked up, almost sticky to touch | |
| Soft moist beginning to loose form | 5 |
| Very soft moist, amorphous | 6 |
| Viscous liquid with some particulate matter | 7 |
| Watery, liquid stools with little particulate matter | 8 |
| Severe watery diarrhea, no particulate matter visible | 9 |

Dogs with a score of 5 and above were enrolled in the study. Fecal scores were determined once a day by a veterinarian on days 1, 2, 3, 4 and 5 of the study for a total of 5 days. All dogs received “standard of care” treatment in addition to SB 300. Such standard of care treatment is described in Example 1. Primary clinical endpoints included fecal consistency and duration of diarrhea; secondary endpoints included safety, dehydration status and attitude/activity of the study animals.

The below table presents the results of the dog-treatment study, which showed that there was a reduction in fecal scores from day 1 to day 5 that was significant between the treatment group that received the enteric coated SB 300 treatment and the group that received placebo (P=0.0413, ANOVA F Test).

| | Reduction in Fecal Score |
|---|---|
| Group | Day 1 to Day 5 |
| SB 300 enteric coated treatment group | 3.583 |
| Placebo | 2.833 |

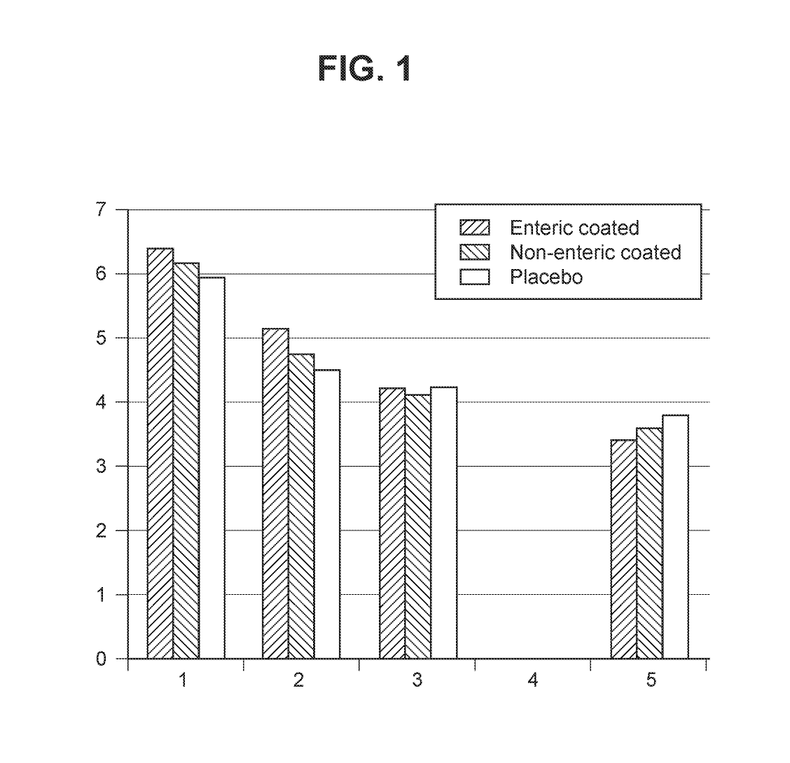

The study described in this Example demonstrated that there was a significant difference in the reduction of fecal scores between day 1 and day 5, thus indicating that SB 300 was efficacious in the treatment of diarrhea. See, FIG. 1. The results of the study further showed that an enteric coating was associated with activity of the SB 300 C. lechleri proanthocyandin polymer composition used for treating the study animals. Results of the study supported the effective treatment of secretory/watery diarrhea (small bowel diarrhea) compared with other types of diarrhea. Additional conclusions drawn from the study were that self-resolving disease required observation of the dogs more often than once a day to identify differences between groups and that the dogs required hospitalization or localization in shelters and/or rescue centers in order to control the collection of data.

### Example 4

Chewable Solid Oral Formulation Containing C. lechleri Proanthocyandin Polymer Composition

A representative chewable, solid, oral dosage formulation, which comprises enterically coated SP 303 microparticles (beads), is presented in this Example. For administration to animals, such as dogs, and as noted hereinabove, the formulation is provided orally and is chewed and swallowed by the animal being treated. The soft chewable solid formulation containing enteric coated SP 303 beads includes the following components, and is not intended to be limiting:

| Component | Amount (g) |
|---|---|
| SP 303 enteric beads | 12.0 |
| Corn starch | 37.5 |
| Dicalcium Phosphate | 22.5 |
| Omalass | 259.8 |
| Artificial Powdered Beef | 60.0 |
| Flavor, PC-0125 | |
| Magnesium Stearate | 7.5 |
| Sugar, Granular | 75.0 |
| Cake Shortening | 7.5 |
| Glycerin, USP | 110.0 |
| Menhaden Fish Oil | 7.5 |
| Soybean Oil | 75.0 |
| Tenox 20 | 0.7 |
| Polyethylene Glycol | 75.0 |

Illustratively, the soft chewable formulation is a 2.5 g soft chew for dogs. The omalass ingredient is dried molasses used as a sweetener.

The chewable solid oral formulation administered as a placebo in the study contained all of the same ingredients as listed for the above formulation, except that the SP 303 enteric beads (active ingredient) were not present, and the omalass ingredient was used in an amount of 271.8 g.

A representative enteric formulation of microparticles (beads) comprising C. lechleri proanthocyanidin polymer/polymer composition (e.g., SB 300 or SP 303) for use in the studies as described in the Examples herein is provided:

| Component | % w/w | g/batch |
|---|---|---|
| Drug-containing layer | | |
| SP 303 or SB 300 | 23.0 | 229.8 |
| Hydroxypropylmethyl | 1.1 | 11.5 |
| cellulose (HPMC) | | |
| Microcrystalline cellulose | 45.9 | 458.7 |
| (MCC) spheres | | |
| Seal coat | | |
| HPMC | 1.3 | 12.7 |
| Triethyl citrate | 0.1 | 1.3 |
| Enteric coat | | |
| Eudragit ® L30D55 | 17.9 | 178.5 |
| Triethyl citrate | 1.8 | 17.9 |
| Talc | 8.9 | 89.3 |
| Total | 100.0 | 999.6 |

As appreciated by the skilled practitioner, the purpose of the enteric coat is to allow the active ingredient (API), e.g., SP 303, to survive the low pH of the stomach and pass safely into the intestine where the enteric coat dissolves and the active is released. Preparation of the enteric-coated active ingredient typically involves a three step process, including:
Step 1: A microcrystalline cellulose (MCC) particle is coated with the active ingredient. Preferably, a minimum of 50% coating is obtained.
Step 2: A sub coat is applied. The sub coat does not add much weight to the overall bead, but serves to prepare the MCC particle for the enteric coating.
Step 3: An enteric coating (top coat) is applied. The enteric coating typically accounts for a 30 to 45% weight gain of the particle.
For a final enteric formulation resulting from the process, the applied drug layer (wg on cellets) is 52.6%; the applied subcoat (wg) is 2%; and the applied enteric topcoat (wg) is 40%.

The final bead (particle) weighs approximately 1 g, in which the active ingredient constitutes about 23% of the weight. The 23% value may be used to determine the number of beads that must be added to the final formulation to obtain the desired dose. A target range for % API in a soft chew form ranges from 15% to 70%. In some cases, the % API in the formulation is increased to 50%.

### Example 5

### Introduction

This Example summarizes the results from a randomized, blinded, two-arm, multi-center study (Cana001) that assessed the clinical efficacy of the compound SP-303 in alleviating clinical signs associated with secretory diarrhea in dogs. The Canalevia™ product used in the study is a canine-specific formulation of crofelemer, SP-303, the active pharmaceutical ingredient, which is isolated and purified from the Croton lechleri tree that is sustainably harvested. The study product is an oral, enteric-coated, twice daily, beef-flavored chewable formulation of crofelemer, SP-303, developed for the treatment of secretory or watery diarrhea, and acute forms thereof, as well as chemotherapy-induced diarrhea, or CID, in dogs, without affecting the animal's intestinal motility. The SP-303 product is not absorbed systemically, but acts locally in the gastrointestinal tract. Canalevia™ is safe, as it has been evaluated in a canine model at an at least 50× dose with no significant lesions.

### Study Goal and Overview

The goal of the study was to investigate the treatment group differences in change from baseline fecal consistency and frequency in dogs with watery diarrhea during a three-day exposure to either SP-303 or placebo. This was a proof of concept study with the goal to define endpoint assessments and statistical analyses that inform a trial design to FDA for a pivotal regulatory dog Canalevia™ study. Veterinarians or trained veterinary technicians conducted this blinded, randomized, placebo-controlled, proof-of-concept study over a five-month period using animals obtained through rescue organizations, shelters and from client owners. Thirty-nine dogs were evaluated in the study based on a six-point score of stool formation as described in the fecal score chart below. Dogs were enrolled in the trial if they were determined to have a baseline fecal score of 4 or 5. Dogs with bloody diarrhea (i.e., fecal score of 6) and/or suspicion of parvovirus were excluded. Subsequent to enrollment, each dog was confined and treatment was administered at the beginning of the score confirmation.

### Study Protocol and Data Collection

To be enrolled in the study, dogs had to satisfy the following inclusion criteria: a Fecal Score of 4 or 5 (watery or liquid stools with little or no particulate matter); male or female; intact or sterilized; between 2 months and 12 years of age; weight between 2 and 40 kg; and any breed or mix. Exclusion criteria for this study were as follows: dogs with other medical conditions which, in the opinion of the investigator, would preclude them from being enrolled into the study; Fecal Score of 3 or below; dogs with bloody diarrhea and/or a suspicion of parvovirus; and dogs who had been treated with any prohibited medications within 7 days of enrollment.

Dogs were randomly allocated in a 1:1 ratio to one of two treatment arms. The treatments were SP-303 (crofelemer), (Treatment A), orally dosed at −2 mg/kg BID (actually dosed at 40 mg packet for animals weighing 2 to 20 kg and two 40 mg packets for dogs weighing 20 to 40 kg) and placebo (Treatment B). Treatment administration commenced after the dog met the enrollment criteria of stool formation score equal to 4 (watery, liquid stool) or 5 (severe watery diarrhea), based on a fecal score scale as shown below.

Fecal Score Scale

| Score | Stool Description |
|---|---|
| 1 | Well-formed stools with |
| | slightly moist surface which |
| | leave marks when picked up |
| 2 | Soft or very soft, moist, |
| | amorphous |
| 3 | Viscous liquid with some |
| | particulate matter |
| 4 | Watery, liquid stool with little |
| | particulate matter |
| 5 | Severe watery diarrhea; no |
| | particulate matter visible |
| 6 | Hemorrhagic diarrhea |

Baseline assessments, which included a physical exam, medical history (if it was available), hematology (CBC), chemistry and fecal exam, were performed on each dog. For dogs in shelters, it was planned that six assessments of fecal scores would be taken per day for each of the three treatment days and one additional follow-up day. For dogs enrolled at clinics, data collection could be less, because animals could be released after four treatments if the diarrhea had resolved. Accordingly, the treatment period for shelter dogs consisted of treatments administered every 10 to 12 hours beginning after the first treatment (T₀) and concluded after a total of 6 treatments had been administered. The treatment period for the client-owned dogs was the same as stated above with the exception that the treatment could be concluded after the second treatment block (i.e., 4 treatments) if the diarrhea had resolved. During their time on study, dogs were kenneled onsite and provided with water and fed the standard diet offered at the trial site. Standard medical care was provided as necessary.

Each dog was sequestered and dosed twice a day over the course of 3×24 hour treatment periods (blocks) for a total of 6 doses. In accordance with the study, a treatment block is defined as a 24-hour period during which two treatments are administered. The first treatment block begins at the time of the first dose administration (T₀). Doses were administered 10 to 12 hours apart. As noted, dogs enrolled into the study were randomized to either SP-303 (crofelemer) (Treatment A) or placebo (Treatment B), but for the purposes of the analysis, the study evaluators were blinded as to whether the treatment assignments corresponded to treatment with SP-303 or with placebo.

### Assessments and Endpoint Definitions

Fecal scoring endpoints were defined using the chart above. In this study, the lowest score on the fecal scoring scale was a 1 (well-formed, moist stools) and the highest score was a 6 (hemorrhagic diarrhea). An assessment could also be recorded as “no stool” in cases where the dog left no stool for assessment at the scheduled time. Using the drug treatment data, the first dose of test article administration was identified and labeled as time zero (baseline). The last fecal assessment prior to time zero was the baseline fecal score; per-protocol this score was to be either a 4 or a 5. Treatment administration and fecal assessments were both recorded by date and time so that the fecal scores could be assigned a time relative to the first dose. Scores in the first 24 hours following first dose were grouped in Block 1 (Day 1); hours 25 to 48 in Block 2 (Day 2); hours 49 to 72 in Block 3 (Day 3). Observation continued from hours 72 to 96 in Block 4 (Day 4).

The evaluator (veterinarian or assistant) conducted the following daily assessments: Fecal scores were obtained at baseline, and every 4 hours during the 3-block treatment period and the observation period using the Fecal Scoring Chart presented above. Episodes of tenesmus or scooting with or without feces production was noted if observed. The dog's general attitude, activity, hydration status, appetite and reaction to abdominal palpation, and body temperature were scored according to scales used in the art.

Time to last unformed stool, TLUS, was calculated from the recorded fecal scores. TLUS was defined as the elapsed time between first dose/time zero and the last unformed stool. Unformed stool was defined in 3 different ways: (1) a fecal score of 2, 3, 4, 5, or 6; (2) a fecal score of 3, 4, 5, or 6; and (3) a fecal score of 4, 5, or 6. If a dog did not have a post-baseline fecal score that was an unformed stool, then TLUS was set equal to zero. In this study, the TLUS definition was restricted to fecal scores in the 72 hours following first dose, which is the completion of the dosing period. The proportion of dogs with alleviated signs of acute watery diarrhea was analyzed. Resolution of diarrhea was defined as a fecal score of 1 or 2 at any post-baseline time. Dogs that did not have a score of 1 or 2 recorded were considered not resolved.

Another exploratory responder analysis was considered as well. For each post-baseline day, the fecal scores for each dog were displayed in order by time. The following guidelines were established: (a) scores of 3, 4, 5, or 6 were considered to be loose stools, and (b) scores of 1 or 2 or no stool for 18 hours were considered to be well-formed stools. The scores were examined clinically under this framework, and each dog was coded as a responder or non-responder on each post-baseline day.

The exploratory endpoints considered in this study were as follows:

Resolution of diarrhea: defined as achieving a formed stool (i.e., score of 1 or 2) at any time during the study period.
Responder analysis: defined as achieving a formed stool (i.e., score of 1 or 2) during any 24-hour treatment block during the study period. Measurement taken during treatment and observation period.
Treatment block: A treatment block was a 24-hour time period. There were three treatment blocks in this study. The first treatment block lasted from T₀ to T₂₄ where T₀ is the time of the first dose administration. There was also an observation block that started 12 hours after administration of the final dose and lasted for up to 24 hours.

### Investigational Material and Administration

Two investigational materials were used in this study: active drug (SP-303) and placebo. Both materials were formulated as enteric coated (EC) beads and packaged in packets containing 250 mg of EC beads representing either investigational veterinary product (IVP) or placebo. The IVP packets with 250 mg of EC beads contained 40 mg of the active substance, crofelemer (SP-303). Each packet of active drug and placebo were assigned a treatment specific code. The distinction between IVP and placebo packet was blinded. Each packet was labeled with a number for randomization purposes. The sites were instructed to assign a packet to a dog based on the sequential entry of each dog at each site. The final formulation of IVP consisted of ingredients considered safe for use with animals. The placebo contained the same formulation ingredients as in the test material minus the SP-303 active ingredient.

Each dose was orally administered based on the weight of the dog at ˜2-4 mg/kg in a Canine Treat, such as, without limitation, GREENIES PILL POCKET® Allergy Formula. Dogs weighing <20 kg received one (1) packet per treatment for a total of six (6) packets. Dogs weighing >20 kg, but <40 kg received two (2) packets per treatment for a total of twelve (12) packets.

### Fecal Scores Analysis

Data for 48 dogs were initially collected in the study: 24 dogs on Treatment A (SP-303), and 24 dogs on Treatment B (placebo). After 48 dogs had completed the study, the sponsor made the decision to suspend the blinded portion of the study with the intent to evaluate the data. The analysis population for the endpoint parameters was the Evaluable Population (per protocol) and defined as all dogs who had completed the study and had sufficient fecal score assessments in the treatment phase and the observation phase for endpoint analysis. A total of 26 investigative sites participated in this study, of which 21 enrolled a total of 48 dogs. Of the 48 dogs enrolled, 39 were included in the Evaluable Population. Nine dogs, designated as numbers 067, 128, 130, 145, 146, 175, 199, 205, 207, were excluded from the analysis due to unreliability in the data collection and recording of observations. Therefore, a total of 39 dogs were analyzed, 23 dogs on Treatment A (SP-303), and 16 dogs on Treatment B (placebo). Dog 001, which was included in this analysis, did not have a baseline fecal score recorded in the database. A note from the site states, “her fecal score has varied between 4 and 5 all weekend”. First dose for this dog was on a Monday so this was a pre-treatment observation.

The mean baseline fecal score in both treatment groups was 4.2. Only one dog in the analysis was below the per-protocol entry requirement. Dog 038 was enrolled per-protocol with a fecal score of 4, although just prior to first treatment, a score of 3 was recorded. Therefore, this dog was recorded with a baseline score of 3. The proportion of dogs with alleviated signs of acute watery diarrhea was analyzed. Resolution of diarrhea was defined as a fecal score of 1 or 2 at any post-baseline time. Dogs that did not have a score of 1 or 2 recorded were considered not resolved.

### Time to Last Unformed Stool (TLUS)

Dogs on Treatment A had a shorter median TLUS than those receiving Treatment B using all 3 methods. Using the log-rank test to compare the Kaplan-Meier plots, statistical significance was approached, but not crossed for 2 of the methods, Method 1 and Method 2. Treatment A appeared to perform better under a stricter requirement for a formed stool.

Time to Last Unformed Stool (72 hours)

| Treatment | Method 1 | Method 2 | Method 3 |
|---|---|---|---|
| Treatment A Median (h) | 56.0 | 42.0 | 24.2 |
| Treatment B Median (h) | 66.0 | 56.3 | 28.5 |
| Log-Rank P-Value | 0.065 | 0.070 | 0.706 |

Note:
In Method 1, an unformed stool was a fecal score of 2, 3, 4, 5, or 6.
In Method 2, an unformed stool was a fecal score of 3, 4, 5, or 6.
In Method 3, an unformed stool was a fecal score of 4, 5, or 6.

### Resolution of Diarrhea

Using a definition of diarrhea resolution being a fecal score of 1 or 2 at any post-baseline time, 21 of 23 (91.3%) dogs treated with SP-303 (Treatment A) responded. This contrasts with dogs treated with placebo (Treatment B), where 8 of 16 (50.0%) of dogs responded. These response rates support the conclusion that a larger proportion of dogs on SP-303 (Treatment A) responded as compared to placebo (Treatment B). The two-sided p-value from Fisher's Exact test was 0.0073.

### Clinical Responder Evaluation

Under the framework described above under “Endpoint Definitions,” each dog was coded as a responder or a non-responder on each day. Dog 202 (Treatment B) was not included in the analysis as the data were too sparse to make a determination. By a similar rationale, Dog 001 was unevaluable on Day 2.

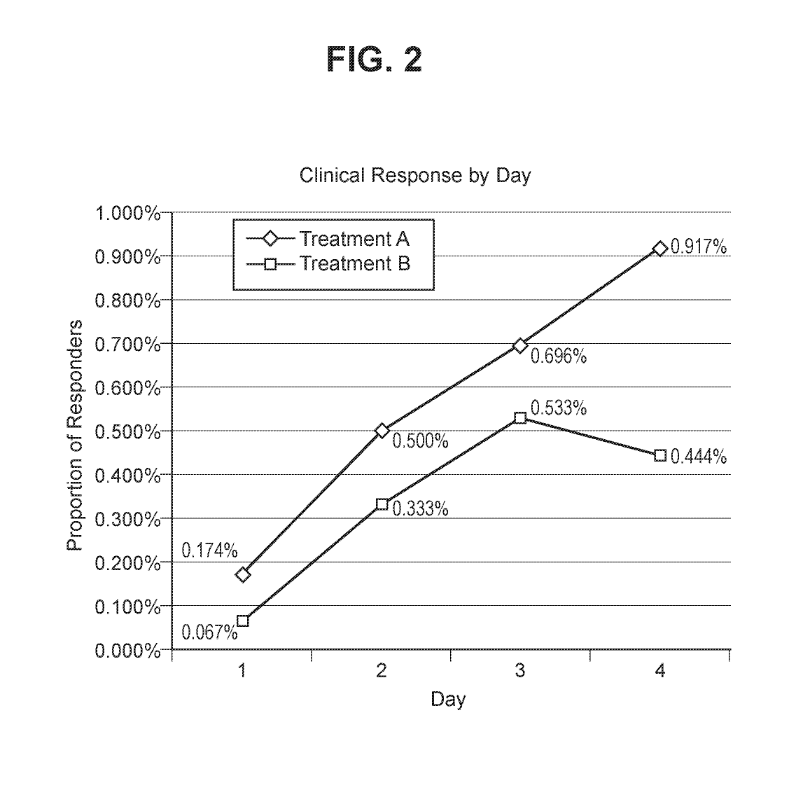

As seen in FIG. 2 and in the table below, the response of dogs receiving Treatment A (SP-303) was greater than that of dogs receiving Treatment B (placebo) during all treatment blocks by at least 10%. A responder is a dog who had formed stools with no follow up unformed stool, day by day. Using a Fisher's Exact test, a significant difference occurred after the third treatment block, the observation period (Day 4). Due to lack of follow-up, the sample sizes on Day 4 were about half of the other days.

All dogs received 6 doses during the treatment period. However, not all dogs were evaluated beyond Block 3 (i.e., during observation phase). It should be noted that only those dogs with fecal scores reported more than 72 hours from T₀ were included in the statistical analysis of the observation phase (T₇₃ to T₉₆).

A Cochran-Mantel-Haenszel test stratified by day provided evidence that the clinical response of dogs receiving Treatment A was greater than that of dogs receiving Treatment B (p=0.013). Using a Fisher's Exact test, a significant difference occurred after the treatment period, on Day 4 (p=0.046). However, because the same dogs appeared in each of the daily evaluations, an alternative model was also considered. A logistic repeated measures model was fit which accounts for the correlations within each dog. This model was suggestive of significance across the post-treatment period with a p-value of 0.059.

Clinical Response by Day
Based on a 24 hour treatment period

| | Treatment A | Treatment B | |
|---|---|---|---|
| Treatment | N = 23 | N = 16 | 2-sided |
| Block (Day) | n (%) | n (%) | P-Value |
| 1 (T₀ to T₂₄) | 4/23 (17.4%) | 1/15 (6.7%) | 0.630^{a} |
| 2 (T₂₅ to T₄₈) | 11/22 (50.0%) | 5/15 (33.3%) | 0.500^{a} |
| 3 (T₄₉ to T₇₂) | 16/23 (69.6%) | 8/15 (53.3%) | 0.492^{a} |
| 4 (Observation)— | 11/12 (91.7%) | 4/9 (44.4%) | 0.046^{a} |
| (T₇₃ to T₉₆) | | | |
| Last Evaluation^{d} | 19/23(82.6%) | 8/15 (53.3%) | 0.073^{a} |
| Comparison of | | | 0.013 |
| proportions across all | | | |
| study^{b} | | | |
| Comparison of | | | 0.059 |
| repeated evaluations^{c} | | | |

Note:
Scores of 4, 5, or 6 were considered to be loose stools/non-responder and scores of 1 or 2 or no stool for 18 hours were considered to be well-formed stools/responder, as the mechanism of action does not create constipation of stool.
^{a}From Fisher's Exact test comparing response rates for each day.
^{b}From CMH test stratified by day.
^{c}From logistic repeated measures model.
^{d}“Last Evaluation” includes data from Observation period and Treatment Block 3 for those dogs that did not have observation scores.

Using a last observation carried forward analysis yielded responses of 83% and 53% for Treatments A and B, respectively. This gives a two-sided Fisher's exact p-value of p=0.073. However, the odds ratio is 4.2, and this converts to a Cohen's d effect size of 0.79, which is considered large. Responder analysis successfully served as the primary endpoint of the study.

This analysis demonstrates the persistence of the effect, with dogs having more days with diarrhea resolution than without diarrhea, as indicated by an analysis of the separation of the curves. The Fischer's Exact test acknowledges that the animals may be exposed to additional causes of watery or secretory diarrhea during the course of the study. The Cochran test acknowledges that any dog that is a responder during treatment block 1 is more likely to be a responder during treatment block 2 and beyond.

### CONCLUSION

This study evaluated the efficacy of dogs randomized to 2 treatment arms, SP-303 and placebo, in a blinded fashion. A simple resolution definition of any post-baseline fecal score equal to 1 or 2 supports the conclusion that Treatment A (SP-303 treatment) is superior to Treatment B (placebo), (p=0.007). Comparison of time to last unformed stool within 72 hours where an unformed stool is defined as fecal score of 3 to 6, approached statistical significance. Finally, using clinical judgment and a criteria where fecal scores of 1 and 2 and no stools for 18 hours were responders, Treatment A was superior on all days and approached significance on Day 4, although follow-up was not available for all dogs through Day 4.

Additionally, using clinical judgment and a criteria where fecal scores of 1 and 2 and no stools for 18 hours were considered responders, Treatment A was superior on all 24-hour treatment periods. It is noted that evaluations were not available for all dogs through the observation phase (post treatment), with a comparison of proportion of responders across the entire study indicating Treatment A, crofelemer, being superior (p=0.013).

The study included all comers and all causes of watery diarrhea allowed within the enrollment criteria. Since it is expected that the observation times could be longer at shelters and with a larger total enrollment study size, it would be advantageous to further explore the shelter and clinic subgroups. Other subgroups, such as diarrhea etiology, diet, and feeding patterns, may be considered as well.

The results obtained from the study described in this example support the conclusion that SP-303 treatment is superior to placebo, with 91% of the SP-303-treated dogs achieving a formed stool during the study versus 50% of the placebo-treated dogs.

The results obtained from this study further support the anti-secretory properties and mechanism of action of SP-303 as described herein above. The SP-303 product acts locally in the gut and is minimally absorbed systemically. It does not alter gastrointestinal motility, has no significant effects on normally functioning intestinal ion channels and electrolyte or fluid transport, and has no side effects different from placebo. These features are further augmented by its lack of effects on the absorption and/or metabolism of co-administered chemotherapy drugs, orally or by other routes of administration. SP-303 acts by normalizing the flow of excess ions and water in the intestinal lumen. Because the flow of excess ions and water into the intestinal lumen is the last step common to the manifestation of acute diarrhea, SP-303 (Canalevia™) offers effective treatment of acute diarrhea in dogs, regardless of cause, including CID.

All patents, patent applications and publications referred to or cited herein are hereby incorporated by reference in their entireties for all purposes.

It is understood that the embodiments and examples described herein are for illustrative purposes and that various modifications or changes in light thereof will be suggested to persons skilled in the pertinent art and are to be included within the spirit and purview of this application and scope of the appended claims. It is to be understood that suitable methods and materials are described herein for the practice of the embodiments; however, methods and materials that are similar or equivalent to those described herein can be used in the practice or testing of the invention and described embodiments.

What is claimed is:

1. A method of treating secretory or watery diarrhea in a non-human companion animal, the method comprising orally administering to the animal in need thereof an enteric or non-enteric coated composition comprising aqueous soluble proanthocyanidin polymer from Croton lechleri in a chewable and flavored solid dosage form and in an amount effective to treat the diarrhea in the companion animal.

2. A method of treating chemotherapy-induced diarrhea (CID) in a canine companion animal, the method comprising orally administering to the animal in need thereof an enteric or non-enteric coated composition comprising aqueous soluble proanthocyanidin polymer from Croton lechleri in a chewable and flavored solid dosage form and in an amount effective to treat the CID in the companion animal.

3. The method according to claim 1 or claim 2, wherein the companion animal is a juvenile or young companion animal.

4. The method according to claim 1 or claim 2, wherein the companion animal is an adult companion animal.

5. The method according to any one of claim 1, 3, or 4, wherein the companion animal is a dog or a cat.

6. The method according to any one of claims 1 to 5, wherein the companion animal is a dog.

7. The method according to any one of claim 1, 3, 4, or 5, wherein the companion animal is a cat.

8. The method according to any one of claims 1 to 7, wherein the C. lechleri proanthocyanidin polymer composition is administered in an amount of 0.5 mg/kg to 25 mg/kg.

9. The method according to claim 8, wherein the C. lechleri proanthocyanidin polymer composition is administered in an amount of 2 mg/kg to 4 mg/kg.

10. The method according to claim 9, wherein the C. lechleri proanthocyanidin polymer composition is administered in an amount of 2 mg/kg twice daily.

11. The method according to claim 10, wherein the C. lechleri proanthocyanidin polymer composition is administered for three days.

12. The method according to claim 9, wherein the C. lechleri proanthocyanidin polymer composition is administered in an amount of 4 mg/kg twice daily.

13. The method according to any one of claims 1 to 12, wherein the companion animal is afflicted with secretory/watery diarrhea, acute diarrhea, episodic diarrhea, or chemotherapy-induced diarrhea.

14. The method according to any one of claims 1 to 13, wherein the chewable solid dosage form of the C. lechleri proanthocyanidin polymer composition is a tablet, pill, or capsule.

15. The method according to any one of claims 1 to 14, wherein the chewable solid dosage form is a soft chewable solid form.

16. The method according to any one of claims 1 to 15, wherein the chewable solid dosage form of the C. lechleri proanthocyanidin polymer composition is beef-flavored.

17. The method according to any one of claims 1 to 16, wherein the chewable solid dosage form comprises polymeric microparticles or nanoparticles containing the C. lechleri proanthocyanidin polymer composition.

18. The method according to claim 17, wherein the polymeric microparticles or nanoparticles are pH-sensitive.

19. The method according to any one of claims 1 to 18, wherein the C. lechleri proanthocyanidin polymer as active ingredient is administered to the animal in a total dosage amount of at least 20 mg to 50 mg.

20. The method according to claim 19, wherein the C. lechleri proanthocyanidin polymer as active ingredient is administered to the animal in a total dosage amount of 40 mg.

21. The method according to any one of claims 1 to 20, wherein the symptoms or health risks associated with the secretory/watery diarrhea or CID in the companion animal include one or more of dehydration, electrolyte imbalance, electrolyte loss, renal insufficiency; nutritional deficiency, alteration of gastrointestinal transit and digestion; or increased risk of infection.

22. The method according to any one of claims 1 to 21, wherein the C. lechleri proanthocyanidin polymer composition is selected from the group consisting of SB 300, SP 303, crofelemer, or a C. lechleri botanical extract.

23. The method according to claim 22, wherein the C. lechleri proanthocyanidin polymer composition is crofelemer.

24. The method according to claim 22, wherein the C. lechleri proanthocyanidin polymer composition is SP 303.

25. The method according to claim 22, wherein the C. lechleri proanthocyanidin polymer composition is SB 300.

26. The method according to any one of claims 22 to 25, wherein the dose is 40 mg two times per day.

27. The method according to any one of claims 22 to 25, wherein the C. lechleri proanthocyanidin polymer composition is an enteric coated composition.

28. The method according to any one of claims 22 to 25, wherein the C. lechleri proanthocyanidin polymer composition is a non-enteric coated composition.

29. The method according to claim 27 or claim 28, wherein the C. lechleri proanthocyanidin polymer composition is formulated as a tablet, pill, capsule, or powder.

30. The method according to any one of claim 1 or claims 5 to 7, wherein the diarrhea is caused by one or more of bacterial infection, parasitic infection, protozoa infection, bacterial infection, illness, disease, pancreatitis, helminth infection, or dietary indiscretions.
